(19) United States
(12) Patent Application Publication
(10) Pub. No.: US 2010/0317671 A1
(43) Pub. Date: Dec. 16, 2010

(54) XANTHINE-BASED CYCLIC GMP-ENHANCING RHO-KINASE INHIBITOR INHIBITS PHYSIOLOGICAL ACTIVITIES OF LUNG EPITHELIAL CELL LINE

(75) Inventor: Ing-Jun Chen, Kaohsiung City (TW)

Correspondence Address: VOLPE AND KOENIG, P.C., UNITED PLAZA, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/831,544

(22) Filed: Jul. 7, 2010

Related U.S. Application Data
(63) Continuation-in-part of application No. 12/176,607, filed on Jul. 21, 2008, now abandoned.

(30) Foreign Application Priority Data
Jan. 29, 2010 (TW) 099102735

(51) Int. Cl. A61K 31/496; C07D 473/08; A61P 11/06; A61P 9/12

(52) U.S. Cl. 514/252.16; 544/272

(57) ABSTRACT

A pharmaceutical composition for a treatment of an interstitial lung disease is provided. The pharmaceutical composition comprises an effective amount of an active component being one selected from a group consisting of a KMUP compound, a KMUP monoquaternary ammonium salt and a KMUP monoquaternary ammonium complex salt, wherein the KMUP monoquaternary ammonium complex salt is synthesized by the KMUP compound and a carboxylic acid derivative of one selected from a group consisting of a statin, a non-steroid anti-inflammatory (NSAIDs) and an anti-asthmatic drug.

### CROSS REFERENCE TO RELATED APPLICATION

This application is a continuation of U.S. patent application Ser. No. 12/176,607, filed Jul. 21, 2008, which is incorporated by reference as if fully set forth.

### FIELD OF THE INVENTION

The present invention relates to a pharmaceutical utility in lung diseases of the KMUP compounds or the KMUP- or piperazine-based quarternary piperazium complex salt.

### BACKGROUND OF THE INVENTION

KMUP-1 (7-[2-[4-(2-chloro benzene)piperazinyl]ethyl]-1,3-dimethylxanthine) as shown in FIG. 1 that obtained from a xanthine derivative with a theophylline backbone where the N-7 position is modified, is a compound having a pleitropic activity that conceived by the inventor. KMUP-1 is known to activate the endothelial nitric oxide synthase (eNOS) in epithelium and endothelium, partially activate the soluble guanynyl cyclase (sGC) and inhibit the phosphodiesterase (PDE). It has been proved that KMUP-1 can influence the cyclic adenosine monophosphate (cAMP)/protein kinase A (PKA) and the cyclic guanosine monophosphate (cGMP)/protein kinase K (PKG) pathways, and result in the increase of the generation of nitrous oxide (NO) in tracheal epithelial cells, thereby activate the intracellular sGC in tracheal smooth muscle cells. Alternatively, KMUP-1 directly activates the intracellular sGC in tracheal smooth muscle cells to increase the amount of cGMP for activating PKG. KMUP-1 may also activate the adenylate cyclase (AC) to induce the increasing amount of cAMP for activating PKA, and both of the PKA and PKG cause the opening of the potassium ion channels in the smooth muscle cells and thus cause the relaxation of the smooth muscle cells (Wu et al., 2004). CAMP and cGMP are secondary messengers in the cells for simultaneously regulating various physiological reactions including cell growth and differentiation, cell apoptosis, glycolysis, lipolysis, and immune and inflammatory reactions. There is study that indicates the KMUP-1 not only induces the releasing of the intrinsic NO, but also has the pharmacological effect similar with the NO donor. Thereby, the applicant filed the applications regarding the activities of KMUP-1 such as anti-hypertension (TW application No. 096121950), the activity for treating prostatic hyperplasia (TW application No. 095112923) and anti-pulmonary hypertension (TW application No. 094129421).

Statins are currently the most therapeutically effective drugs available for reducing the concentration of the low-density lipoprotein (LDL) particle having the risk of cardiovascular disease existing in the bloodstream of patients. US. Patient No. 7,390,504 discloses a water soluble salt formula consisting of a dihydroxy open-acid statin and a fibric acid. However, Evans M. et al. has found that statin incurs the untoward effects of myotoxicity and rhabdomyolysis, especially utilized in combination with the fibric acid drugs (Drug Saf. 2002). The recent study of Jacobson TA. can not overcome the above-mentioned risk as well although it is considered that there has been none of the data obtained from a large-scale experiment for supporting the risk resulted from combing the statin and the fibric acid drugs (Nat Rev Endocrinol. 2009).

### SUMMARY OF THE INVENTION

The idiopathic pulmonary fibrosis (IPF) is a progressive inflammatory lung disease. Over 200 kinds of interstitial lung disease (ILD) may cause the IPF and the common reasons include the infection, drug and the autoimmune disease. There is still no clear epidemicology incidence report in the world and the most of the reports are local statistical data. Until now, the most IPF are idiopathic and have a poor prognosis that the patients usually die in 5-6 years after the diagnosis.

Clinically, the patients usually show the chonic dry cough, wheeze, marasmus or abnormal breath sound. The abnormal expressions may be observed in the chest X-ray and the lung function of the patients are mostly restrictive lung diseases occasionally with the tracheal obstruction problem. Nevertheless, the computed tomography and the pathological tissue section are dominant analyses for aiding the diagnosis. Under the microscope, the changes of the chronic inflammation, the type II alveolar cell proliferation, the vascular endothelium proliferation, the interstitial structure reconstruction and the fibrosis can be observed.

The developing process of the IPF disease accompanying with the inflammatory response and interstitial cell proliferation has been discovered in the past years. When a stimulant enters the lung, it will firstly cause the damages of the lung epithelial cells or vascular endothelium, destroy the normal structure to generate the inflammation, induce the cytokine dysregulation and release a large amount of inflammatory cytokines. Then, the anti-fibrotic and pro-fibrotic cytokines will increase, which are in an equilibrium state under normal condition. The minor damage can be repaired by angiogenesis and scavenging of excess extracellular matrix. However, the servere damage or the pro-fibrotic cytokine overexpression will cause the fibrosis of lung. During the IPF process, the inflammation and many regular cytokines such as cytokines, kemokines and growth factors released by the interstitial cells result in the migration and proliferation of the fibroblast and secretion of more extracellular matrix. The lung tends to being fibrosis when the excess extracellular matrix cannot be scavenged or there is a vascular formation problem. Simply speaking, there is still no therapeutic drug in clinical for IPF that includes acute or chronic lung inflammation resulting in inreversable lung damage.

The inventor has conceived of preparing the monoquaternary piperazium complex salt as

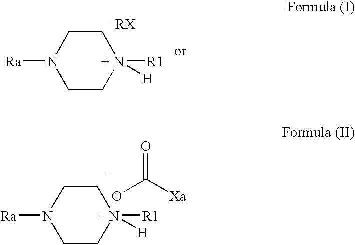

with KMUP compounds or piperazine by chemical synthesis.

The synthesis of monoquarternary piperazium complex salt is performed by mixing the KMUP compounds with a mixing solution of C₁-C₄ lower alcohol and water, and reacting the mixture with the sufficient amount of the mineral acid or organic acid to form the monoquarternary ammonium salts. Additionally, the monoquarternary ammonium salts of the mineral salt or the organic salt of the MUP compounds are mixed with a mixture of C₁-C₄ lower alcohol and water, wherein the amount of the KMUP compounds is sufficient to dissolve the reactant of the “RX” group such as the reactant having a carboxyl group including the carboxylic derivative of the statin, the ester derivative of the statin, the statin derivative with the protecting group, the NSAIDs, prostacyclin (PGI₂), the anti-asthma drugs, Repaglinide and Nateglinide, Montelukast, Cromolyn sodium, Nedocromil, Gemfibrozil and Bezafibrate. The C₁-C₄ lower alcohol is chosen and the amount of the mixing solution is adjusted upon factors such as the proportion of water, the reacting temperature and the purity of the statin ester derivative. Preferred alcohols are ethanol and isopropyl alcohol (IPA) with 5% to 30% water, more preferably 10% water and about 90% ethanol or IPA. The statin ester derivative is hydrolyzed in the base catalyst, and which is added in the mixing solution in an amount about 10 mmoles L⁻¹ to about 1 mole L⁻¹. The temperature of the mixing solution should be heated to about 40° C. to 70° C. in sequence to reflux the mixing solution for accelerating the reaction. The resulting KMUP-1 monoquarternary piperazium complex salt should be re-dissolved in the mixing solution after being filtered, and preferably being re-crystallized under room temperature. The above-mentioned mineral acids include hydrochloric acid (HCl), hydrobromic acid (HBr), hydroiodic acid (HI), sulfuric acid (H₂SO₄), nitric acid (HNO₃), phosphoric acid (H₃PO₄), sodium dihydrogen posphate (NaH₂PO₄) and disodium hydrogen phosphate (Na₂HPO₄). The organic acids are selected from a group consisting of citric acid, glycyrrhizic acid, fumaric acid, maleic acid, nicotinic acid, isonicotinic acid, tartaric acid, succinic acid, adipic acid, fatty acid, methanesulfonic acid and phenoxylevulinic acid. The above contents are disclosed in TW patent application having the application No. 099102735 filed on Jan. 29, 2010.

KMUP-1, KMUP-2 and KMUP-3 are referred as KMUP or KMUPS, and the monoquarternary ammonium salts such as KMUP mineral acid salt or the organic acid salt thereof are ordinarily referred as KMUP compound and the exception thereof will be especially marked out in the specification. The monoquarternary piperazium complex salt synthesized by KMUP compound together with the statins containing a carboxyl group, the fibric acid derivatives for lipid lowering, the anti-inflammatory drug containing a carboxyl group, the amino acids or the acetylcysteine containing a carboxyl group or the anti-asthmatic drug containing a carboxyl group, as the monoquarternary piperazium complex salt synthesized by KMUP compounds of Formula (I) or Formula (II) or the piperazine, where R₁ and Rₐ being hydrogen refers to a piperazine complex salt and R₁ and Rₐ being not hydrogen refers to a KMUP compounds of KMUP-1, KMUP-2 and KMUP-3. For example, the KMUP-2HCl salt is 7-[2-[4-(2-methoxybenzene)-piperazinyl]ethyl]-1,3dimethylxanthine HCl and KMUP-3 HCl salt is 7-[2-[4-(4-nitrobenzene)-piperazinyl]ethyl]-1,3-dimethylxanthine HCl, which belong to chlorobezyl piperazine-based monoquarternary piperazium complex salt as shown in FIG. 1.

The above RX group may be selected as a mineral acid, an organic acid, the statins containing a carboxyl derivative, the fibric acid derivatives for lipid lowering, the anti-inflammatory drug containing a carboxyl group, the amino acids or the acetylcysteine containing a carboxyl group or the anti-asthmatic drug containing a carboxyl group, and RX⁻ may be an anion of the above-mentioned groups carrying a negative charge. The above-mentioned halogen refers to fluorine, chlorine, bromine and iodine. The anti-inflammatory drug containing a carboxyl group refers to the related drugs for inhibiting human immune system including NSAIDs containing a carboxyl group in structure and anti-asthma drug, if necessary.

Formula (I) may represent as

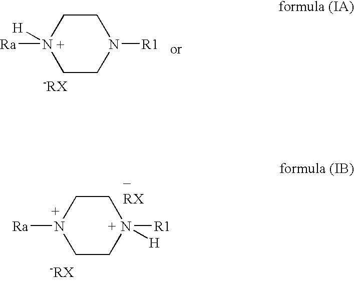

with carboxylic dimers according to the amount of the reacting acid and stereo combination. When a partial structure except the carboxyl group in the organic acid, the statin containing a carboxyl derivative, the fibric acid derivative for lipid lowering, the NSAIDs containing a carboxyl group, the amino acids or the acetylcysteine containing a carboxyl group, and the anti-asthmatic drug containing a carboxyl group are represented as Xa, formula (I) may show as formula (II), the formula (IA) may show as

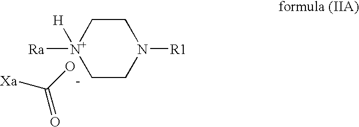

and formula (IB) may show as

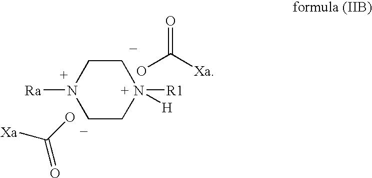

In view of the drawbacks of current techniques, the inventor conceives the present invention “XANTHINE-BASED CYCLIC GMP-ENHANCING RHO-KINASE INHIBITOR INHIBITS PHYSIOLOGICAL ACTIVITIES OF LUNG EPITHELIAL CELL LINE” for overcoming the drawbacks of the prior art. The summary of the present invention is described below. The present invention is a serial invention of the TW patent application having the application No. 099102735 filed on Jan. 29, 2010.

According to the present invention, the monoquarternary ammonium salts synthesized by reacting the KMUP compound or piperazine selectively with one of the mineral acid and organic acid, the monoquarternary piperazium complex salts of formula (I) or formula (II) synthesized by reacting the KMUP compound or piperazine selectively with a derivative containing a carboxyl group in structure (such as the statins, the fibric acid derivatives for lipid lowering, the NSAIDs, the NO-increasing amino acid arginine, the mucolytic agent cetylcysteine, the anti-asthma drug, the PGI₂, or the KMUP compound itself may be utilized in asthma, and the lung interstitial disease caused by inflammation, IPF, acute or chronic lung hypertension. The above statins may be commercial available statin drugs, including Atorvastatin, Cerivastatin, Fluvastatin, Lovastatin, Mevastatin, Pravastatin, Rosuvastatin, Simvastatin and Pramastatinic acid.

The above anti-inflammatory drugs may be the commercial NSAIDs and usually have a carboxyl group in the structure, such as Aspirin, Salicylic acid, Indomethacin, Diclofenac, Meclofenamic acid, Tolmetin, Ketoprofen, Glycerrhizic acid, Flurbiprofen, Fenoprofen, Tiaprofen, Diflunisal, Etodolac, Ibuprofen, prostacyclin and Zhankuic acids A, B and C, and Etodolac. The above anti-asthma drug is selected from the commercial drugs such as Montelukast, Cromolyn sodium and Nedocromil.

According to the present invention, the KMUP compound itself and the monoquarternary piperazium complex salts of formula (I) or formula (II) synthesized by KMUP compound or piperazine may become a pharmaceutical composition by adding an appropriate amount of excipients, which can be processed by formulation as various formulas for administering to the mammals and show the therapeutical function for the lung interstitial disease as described above.

The complex salt prepared in accordance with KMUP compound itself and the embodiments and the monoquarternary piperazium complex salts of formula (I) or formula (II) synthesized by KMUP compound or piperazine are assayed in an IPF animal model induced by bleomycin. In the 28-day experimental process, the 7^{th} day after the bleomycin administration is served as an interface between the early inflammatory stage and late fibrotic stage. The inflammatory cells and the amount of the collagen in the lavage fluid are analyzed to reflect the fibrotic formation. The lung damage induce the inflammation and inflammatory cells aggregation, which cause the unbalanced distribution of the various metalloprotease and metalloprotease inhibitor in extracellular matrix in the lung tissue and the aggregation of the excess collagen via the effects of the peroxide ion, cytokines and growth factors, and thus form the fibrosis.

In the observation of the early inflammatory stage, the amount of inflammatory factor in the lung lavage fluid and the expressions of MMP-2 and MMP-9 are measured, and the aggregation of the inflammatory factor is observed by tissue histology. In the late fibrotic stage, the amount of the collagen in the lung tissue is measured, and the fibrosis accumulation and the location thereof are observed by tissue histology. Additionally, western blotting is utilized to survey the protein expression in the lung tissue. It is expected for the IPF treatment by confirming whether the monoquarternary piperazium complex salts of formula (I) or formula (II) inhibit the inflammation and fibrosis formation by activating eNOS and inhibiting the HMGB1/TGF-β/Smad3 related pathway.

In the mice administered with bleomycin, the results show that the amount of white blood cell (WBC) in lung lavage is increased, the expressions of the matrix metalloproteases MMP2 and MMP-9 are significant and the expression of the pro-inflammatory factor HMGB1 is increased at day 7. At day 28, it is observed that expression of eNOS is decreased, the expressions of TGF-β, Smad 3 and HMGB1 are increased and there is significant collagen accumulation in the tissue sections. The KMUP-1 and KMUP-5 administrations can efficiently decrease the amount of WBC and the expressions of MMP2, MMP-9 and HMGB1 in lung lavage during the lung inflammatory stage, and increase the expression of eNOS and inhibit the expressions of TGF-β and Smad 3 during lung fibrotic stage.

Among the KMUP compound itself and the monoquarternary piperazium complex salts of formula (I) or formula (II), the monoquarternary ammonium salt synthesized by one of the mineral acid salt or the organic acid salt of KMUP, or the monoquarternary piperazium complex salt synthesized by reacting the KMUP compound or piperazine selectively with one derivative containing a carboxyl group in structure (such as the statins, the NSAIDs, the amino acids, the acetylcysteine and the anti-asthma drug), may be utilized in asthma, and the lung interstitial disease caused by inflammation, IPF, acute or chronic lung hypertension. According to the present invention, the KMUP compound itself and the monoquarternary piperazium complex salts of formula (I) or formula (II) that is most appropriate for the lung interstitial disease and show the therapeutic effect refer to KMUP-1, KMUP-2, KMUP-3, KMUP-1HCl, KMUP-2HCl, KMUP-3HCl, KMUP-1-Glycerrhizic acid, KMUP-1-Nicotinic acid, KMUP-1-Folic acid, KMUP-1-Folinic acid, KMUP-1-γ-Polyglutamic Acid, KMUP-1-PGI₂, KMUP-1-GLT Co-polymer acid, KMUP-1-Simvastatinic Acid, KMUP-1-Nedocromil, KMUP-1-Montelukast, KMUP-1-Methotrexate, KMUP-2-Glycerrhizic acid, KMUP-2-Nicotinic acid, KMUP-2-Folic acid, KMUP-2-Folinic acid, KMUP-2-γ-Polyglutamic Acid, KMUP-2-PGI₂, KMUP-2-GLT Co-polymer acid, KMUP-2-Simvastatinic Acid, KMUP-2-Nedocromil, KMUP-2-Montelukast, KMUP-2-Methotrexate, KMUP-3-Glycerrhizic acid, KMUP-3-Nicotinic acid, KMUP-3-Folic acid, KMUP-3-Folinic acid, KMUP-3-γ-Polyglutamic Acid, KMUP-3-PGI₂, KMUP-3-GLT Co-polymer acid, KMUP-3-Simvastatinic Acid, KMUP-3-Nedocromil, KMUP-3-Montelukast and KMUP-3-Methotrexate. According to the experiments in the present invention, it is proved that the lung damage induces the inflammation and inflammatory cells aggregation, which cause the unbalanced distribution of the various metalloprotease and metalloprotease inhibitor in extracellular matrix in the lung tissue and the aggregation of the excess collagen via the effects of the peroxide ion, cytokines and growth factors, and thus form the fibrosis.

The above excipient or the phrases “pharmaceutically acceptable carrier or excipient” and “bio-available carrier or excipient” include any appropriate compound known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carrier or excipient will not cause the undesired effect, allergy or other inappropriate effects while being administered to an animal or human. Accordingly, the derivatives disclosed in the present invention in combination with the pharmaceutically acceptable carrier or excipients are adaptable in the clinical use and in the human. A therapeutic effect can be achieved by using the dosage form in the present invention by the local or sublingual administration via the venous, oral, and inhalation routes or via the nasal, rectal and vaginal routes. About 0.1 mg to 100 mg per day of the active ingredient is administered for the patients of various diseases.

The carrier is varied with each formulation, and the sterile injection composition can be dissolved or suspended in the non-toxic intravenous injection diluent or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bioavaliable enhancing agent used for developing the formulation that used in the pharmaceutical industry.

The composition for oral administration adopts any oral acceptable formulation, which includes capsule, tablet, pill, emulsion, aqueous suspension, dispersing agent and solvent. The carrier generally used in the oral formulation, taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention also can be administered intravenously, as well as subcutaneously, parentally, muscular, or by the intra-articular, intracranial, intra-articular fluid and intraspinal injections, the aortic injection, the sterna injection, the intra-lesion injection or other appropriate administrations.

### The Bleomycin-Induced IPF Animal Model

It is known that in the various models using the high-pressure oxygen, the inhalative stimulant, the irradiation, the intraperitoneal injection and the tracheal administration for studying lung fibrosis, the bleomycin-induced IPF through tracheal administration is the most stable and broadly utilized animal model.

Bleomycin is a glycoprotein antibiotic, which is currently used in the therapy for the cancers such as the testicular cancer, the lymphoma, the squamous cell carcinoma in each sites, the head and neck cancer, the cervical cancer, the penis cancer and skin cancer. The dominant mechanism of bleomycin resides in cutting the intracellular DNA by using the dual function in the structure of bleomycin, including the DNA binding site and the active redox site. The redox site may bind with the ferrous ion for generating the intermediate complex, bleomycin-Fe (II)-O₂. The fragments including the single and double stranded DNA are broken when the electron is transported to oxygen by the ferrous ion. It is known the importance of the ferrous ion and the oxygen ion for resulting in the DNA fragments, for example, the high concentrated oxygen may significantly increase the cytotoxicity of the bleomycin in the presence of the ferrous ion while the bleomycin has no toxicity in the absence of the ferrous ion. In the anaerobic environment, the generation of the oxygen free ion is reduced and the bleomycin cannot cleave the DNA. The anti-oxidant N-acetylcysteine (NAC) and bilirubin may decrease the fibrosis induced by bleomycin, which might be related to the decreasing in oxygen free ion generation. Bleomycin is also affected by the bleomycin hydrolase that is discovered in the cancer cells, liver and kidney but apparently absent in lung the epithelium.

These results show that bleomycin will increase the amount of tracheal secretion cells and their mucin generation, and that NAC will improve the lung pathology and shorten the mucin secretion in the bleomycin rat model (Mata, M., A. Ruiz, et al. (2003). “Oral N-acetylcysteine reduces bleomycin-induced lung damage and mucin Muc5ac expression in rats.” Eur Respir J 22(6): 900-905).

Therefore, bleomycin will result in a specific toxicity to lung and skin, and the toxicity of bleomycin in lung will increase by the increasing dosage. It has been proved in clinical that bleomycin make the patient show the phenomenon of lung fibrosis, even make the patient's condition more serious thereby that cannot be prevented and treated. The intratracheal injection of bleomycin in the animal may demonstrate the pathology similar to IPF where the violent inflammation is shown in the early 7 days, the fibrosis formation can be observed on day 14 and the state of the fibrosis is more serious on days 21 to 28. Accordingly, the therapeutic effect of drugs can be evaluated thereby.

### Transforming Growth Factor-β (TGF-β)

TGF-β is an important mediation for stimulating the differentiation of the myofibroblast in both of the repair process of the damage tissue under normal physiology or the pathogenesis of the fibrotic state. TGF-β itself also involves in the cell proliferation, fibrosis, the tissue repair, the inflammation, the apoptosis, the cell differentiation, the cell adhesion and the cell migration. The elevated amount of TGF-β can be discovered in the lung lavage from the patients of the lung fibrosis. However, the animal with the induced IPF also has an elevated amount of TGF-β. The above TGF-β involving pathways may divided into two signaling pathways depending on SMAD gene or non-SMAD gene. SMAD gene refers to the Sma and MAD genes in the mammal. Since the three genes, Sma2, Sma3 and Sma4, in the Caenorhabditis elegans and the Mother Against Dpp gene in Drosophila melanogaster have the sequence homology, the Sma gene and the MAD gene are referred as SMAD gene.

The TGF-β involving pathway mediated by SMAD gene is to phosphorylate and translocate the common-partner-SMAD (co-SMAD) in the nuclear via activating Smad2/3 for regulating the further downstream. The involving pathway mediated by non-SMAD gene relates to the RhoA, PI3k and MAPK signaling transductions. Both of the SMAD or non-SMAD signaling transductions relate to the fibrosis formation, such as promoting the expression of the connective tissue growth factor (CTGF), promoting the accumulation of the collagen and forming the fibrosis.

### High Mobility Group Box 1 (HMGB1)

HMGB1 is a non-histone chromosomal protein, which can maintain the nucleosome structure, regulate the gene transcription and activate the DNA recombination, repair and replication functions. It is known that HMGB1 is released by activated macrophage and may induce the production of other pro-inflammatory factors. This protein is associated with the generation of the diabetes, the immune response, the inflammatory disease and tumor.

After the bleomycin administration it is found that the aggregation of the inflammatory cells is induced, the HMGB1 expression is increased, the generation of TGF-β and the epithelial-mesenchymal transition (EMT) aggregation is promoted and the fibrosis is formed, so that the HMGB1 is deemed as a upstream promoting factor in the fibrosis progression.

### Matrix Metalloproteinases (MMPs)

In the recent years, the relationship between the expression of extracellular MMPs and tissue damage attracts considerable attention. The oxidative stress and nitrative stress are important reasons for the lung damage, and the MMPs induction plays an important role therein. MMP is a reference index of the IPF, it also plays an important role in the process of lung tissue regeneration and fibrosis. MMP family is a group of zinc- and calcium-dependent endopeptidases, which is an enzyme capable of degrading the matrix and depositing the extracellular matrix (ECM). MMP relates to the inflammation, tissue repair, angiogenesis and wound healing. It is found that the generation of lung fibrosis induced by bleomycin is associated with the increased activities of two gelatinases, MMP-2 and MMP-9. Bleomycin may induce the gene expressions of the MMP in the lung tissue and the macrophage metalloelestase (MME), make the ECM rearrangement in lung tissue out of control and thus promote the lung fibrosis. MMP-9 is dominantly secreted by the inflammatory cells, and the MMP-2 is produced by the structural cells such as fibroblast, the epithelial cell and endothelial cell.

Nitric oxide (NO) is generated from arginine catalyzed by nitric oxide synthases (NOS). It is known that the various NOS, the neural NOS (nNOS), inducible NOS (iNOS) and endothelial NOS (eNOS), have characteristic and function respectively. In the IPF process, the expression of the eNOS is more relative than that of the iNOS.

RhoA belongs to a GTP-binding protein of the Ras family. Rho-kinase (ROCK) is a target protein in the downstream of Rho, which is a serine/threonine enzyme with a molecular weight about 160 kDa, and the two isomers, ROCk I and ROCK II, have been defined. The later is prioritly expressed in the brain and skeletal muscle, and both are expressed in the heart. It is found that sildenafil mitigates the bleomycin-induced IPF through inhibiting the activation of RhoA/ROCK, and the Rho kinase inhibitor Y27632 also shows the anti-fibrosis effect in the animal experiment. Since the pulmonary hypertension symptom also forms the lung fibrosis, it is inferred that the drug for treating the pulmonary hypertension (i.e. the KMUP-1) also can be utilized in IPF.

The endothelin antagonist Bosentan can treat the IPF in clinical. The downstream of the endothelin-1 (ET1) may affect the NF-κB and CTGF and thus relates to the fibrosis formation.

Clarithromycin is a new generation derivative of the erythromycin. There are studies proving that Clarithromycin may regulate the wound healing by inhibiting the migration of the fibroblast. Additionally, there are also studies indicating that the 12-membered ring derivatives of some erythromycins have anti-inflammatory and anti-fibrosis effect both in vivo/in vitro, and the involving pathway is dominantly through inhibiting the nuclear translocation of TGF-β and Smad3. By using the anti-inflammatory effect of the Clarithromycin, it may ease the inflammatory state during the IPF progression in clinical. 10 mg kg⁻¹ day⁻¹ of Clarithromycin is used in the present invention for comparing the anti-inflammatory effect with that of KMUPs.

### Combination Therapy

There is no effective therapeutic drug for IPF, there are only drugs directed to the related indexes evoked in the course of disease can be administered and ease the disease, for example, the antioxidant (N-acetylcystein and endothelin), the receptor antagonist (bosentan), and the TNF-alpha antagonist (etanercept). The inventor assumes that the combination therapy not only inhibits in multiple aspects but decreases the side effects resulted from the dose of the drug. It is proved that the combination of 20 mg kg⁻¹ day⁻¹ simvastatin and 75 mg·kg⁻¹·day⁻¹ sildenafil has the therapeutic effect on the pulmonary hypertension, and the effect is better than using simvastatin or sildenafil alone. Since the KMUP-1 has been proved to have a therapeutic effect on the pulmonary hypertension and the involving molecular pathway thereof is similar with that of sildenafil, a combination of simvastatin and KMUP-1 is adopted in this experiment.

### Experimental Animal Model

The six-week old mice are used in this experiment and divided into ten groups as follows.

| Control group (CTL) | Dose | | administration |
|---|---|---|---|
| Bleomycin (BLM) | 4U | kg⁻¹ | IT on day 0 |
| KMUP-1 | 1, 2.5, 5 | mg kg⁻¹ | On day −3~7 |
| Simvastatin (Sim) | 5 | mg kg⁻¹ | and day −3~28 |
| Simvastatin in | 2.5 | mg kg⁻¹ (each drug) | in average |
| combination with | | | |
| KMUP-1 | | | |
| KMUP-5 | 2.5 | mg kg⁻¹ | |
| Clarithromycin (CAM) | 10 | mg kg⁻¹ | |

(1) The normal group
(2) The pathology group: 4U kg⁻¹ bleomycin is administrated via intratracheal injection on day 0.
(3) The treating group: the drugs are administered 3 days before the bleomycin

administration (FIG. 3).

I. The Body Weight Change of Each Group of Mice

The six-week old male C57B1/6 mice are purchased from the National Laboratory Animal Center. The body weight of the normal control group increases stably and the hair of the mice are shiny. The pathology group shows significant marasmus on day 7 and their body weights are slowly increased after day 7. The marasmus is less significant in the mice administered with KMUP-5.

TABLE 1
The body weight change profile of the IPF mice

| | Body weight (g) | | | |
|---|---|---|---|---|
| | Day −3 | Day 7 | Day 14 | Day 28 |
| CTL | 24.26 ± 8.71 | 25.04 ± 5.56 | 26.58 ± 3.72 | 26.45 ± 1.77 |
| BLM | 23.68 ± 0.98 | 20.58 ± 3.51 | 21.88 ± 4.46 | 22.88 ± 5.1 |
| KMUP₁-1 mg | 24.12 ± 1.96 | 19.80 ± 3.13 | 20.48 ± 5.68 | 21.90 ± 5.27 |
| KMUP₁- | 23.06 ± 1.36 | 17.84 ± 1.72 | 19.11 ± 3.4 | 20.43 ± 2.8 |
| 2.5 mg | | | | |
| KMUP₁-5 mg | 23.13 ± 1.42 | 18.33 ± 1.3 | 19.83 ± 2.3 | 21.13 ± 2.5 |
| Sim | 23.5 ± 1.32 | 17.9 ± 1.56 | 19.8 ± 2.31 | 20.3 ± 1.65 |
| Combination | 23.4 ± 2.33 | 18 ± 1.64 | 20.1 ± 2.31 | 20.4 ± 2.14 |
| KMUP₅ | 23.87 ± 2.04 | 22.25 ± 2.36 | 23.15 ± 2.51 | 23.03 ± 2.74 |
| CAM | 24.6 ± 1.48 | 19.93 ± 3.28 | 19.17 ± 3.77 | 24.7 ± 1.21 |

CTL(control group), BLM (Bleomycin), Sim (Simvastatin), Combination (Simvastatin 2.5 mg/kg in combination with KMUP-1 2.5 mg/kg), KMUP₅ (KMUP-1-Simvastatinic Acid), CAM (Clarithromycin)

II. Tissue Section Observation

1. Hematoxylin and Eosin Staining (H&E Stain)

In the histology observation, the bleomycin-induced mice show the pathology characteristic of lung fibrosis. In the lung tissue sections of the normal control group, the structure of the lung tissue is clear and has no inflammatory cells infiltration, the alveolar wall is complete and not being broaden, the lung atrophy and the vascular wall thickening are not found, and the inflammatory cells infiltration is not found in the bronchial cavity and the alveolar cavity. On 7 days after the bleomycin administration, the alveolar septum is significantly broadened and there are considerable inflammatory cell aggregation in the alveolar septum and alveolar cavity (mostly the monocyte/macrophage). On day 28, the alveolar is dystructured, the macrophage aggregation can be seen in part of aveoli, the fibroblast and fibric cell proliferate, and the fibers are accumulated in the lung interstitium. In the treating group, the inflammatory state is inhibited well on day 7. The clarithromycin group still has a significant inflammation phenomenon on day 28.

2. Masson Trichrome

In the aspect of Masson trichrome, the pathology group has demonstrated the fibrosis aggregation on day 7 and considerable fibrosis aggregation on day 28. There are still partial fibrosis in the simvastatin group and the combination group.

III. The WBC Count Influence

The amounts of the total cell, the neutrophill, the lymphocyte, the monocyte, the eosinophill and the basophill are analyzed by an automatic animal blood cell analyzer for evaluating the extent of the inflammation. As shown in Table 2A, the WBC total number in the lung lavage significantly increases in the pathology group mice after 7 days of the bleomycin administration (as shown in FIG. 4A). As shown in Table 2B, the WBC total number in the pathology group mice is about half of that on day 7 although the WBC total number in the pathology group mice is more than the normal control group (as shown in FIG. 4B). As shown in FIGS. 5A and 5B, the changes of the neutrophill and lymphocyte amounts are most significant. The inflammatory condition is well inhibited in the treating groups on day 7, in which the inhibition has statistical significant (p<0.05).

TABLE 2A
The inflammatory cells in the lung lavage of mice administrated with bleomycin on day 7

| Day 7 | WBC total | NE | LY | MO | EO | BA |
|---|---|---|---|---|---|---|
| CTL | 1.63 ± 0.52 | 0.62 ± 0.2 | 0.35 ± 0.12 | 0.06 ± 0.03 | 0.43 ± 0.15 | 0.22 ± 0.11 |
| BLM | 7.24 ± 1.2 | 1.46 ± 0.31 | 3.82 ± 1.04 | 0.4 ± 0.23 | 1.05 ± 0.35 | 0.51 ± 0.17 |
| KMUP 1 mg | 3.51 ± 0.01 | 0.92 ± 0.13 | 1.83 ± 0.11 | 0.12 ± 0.02 | 0.44 ± 0.02 | 0.2 ± 0.01 |
| KMUP 2.5 mg | 3.2 ± 0.49 | 0.91 ± 0.36 | 1.81 ± 0.09 | 0.07 ± 0.01 | 0.17 ± 0.11 | 0.11 ± 0.02 |
| KMUP 5 mg | 2.43 ± 0.01 | 0.33 ± 0.14 | 1.76 ± 0.01 | 0.07 ± 0.01 | 0.16 ± 0.01 | 0.1 ± 0.02 |
| Sim | 3.09 ± 2.98 | 0.76 ± 0.53 | 1.5 ± 2.17 | 0.13 ± 0.19 | 0.46 ± 0.4 | 0.22 ± 0.23 |
| Combination | 1.090 ± 0.71 | 0.2 ± 0.13 | 0.76 ± 0.49 | 0.06 ± 0.04 | 0.06 ± 0.05 | 0.02 ± 0.01 |
| KMUP₅ | 0.74 ± 0.21 | 0.18 ± 0.08 | 0.45 ± 0.17 | 0.03 ± 0.02 | 0.06 ± 0.04 | 0.02 ± 0.01 |
| CAM | 0.89 ± 0.3 | 0.2 ± 0.09 | 0.56 ± 0.17 | 0.05 ± 0.02 | 0.07 ± 0.02 | 0.02 ± 0.01 |

WBC total (White blood cell total count), NE (neutrophil), LY (lymphocyte), MO (monocyte), EOS (eosinophil), BA (basophil)
CTL (control group), BLM (Bleomycin), Sim (Simvastatin), Combination (Simvastatin 2.5 mg/kg in combination with KMUP-1 2.5 mg/kg), KMUP₅ (KMUP-1-Simvastatinic Acid), CAM (Clarithromycin)

TABLE 2B
The inflammatory cells in the lung lavage of mice administrated with bleomycin on day 28

| Day 28 | WBC total | NE | LY | MO | EO | BA |
|---|---|---|---|---|---|---|
| CTL | 1.77 ± 0.61 | 0.64 ± 0.22 | 0.39 ± 0.13 | 0.06 ± 0.03 | 0.47 ± 0.18 | 0.22 ± 0.11 |
| BLM | 3.62 ± 1.2 | 0.73 ± 0.31 | 1.91 ± 1.04 | 0.2 ± 0.23 | 0.52 ± 0.35 | 0.26 ± 0.17 |
| KMUP 1 mg | 3.41 ± 0.54 | 0.61 ± 0.11 | 1.89 ± 0.76 | 0.2 ± 0.25 | 0.42 ± 0.17 | 0.21 ± 0.06 |
| KMUP 2.5 mg | 1.95 ± 0.92 | 0.36 ± 0.16 | 1.24 ± 0.43 | 0.07 ± 0.02 | 0.17 ± 0.12 | 0.11 ± 0.7 |
| KMUP 5 mg | 1.8 ± 0.01 | 0.33 ± 0.14 | 1.1 ± 0.01 | 0.07 ± 0.01 | 0.16 ± 0.01 | 0.1 ± 0.02 |
| Sim | 3.09 ± 0.92 | 0.76 ± 0.61 | 1.5 ± 3.03 | 0.13 ± 0.26 | 0.46 ± 0.35 | 0.22 ± 0.22 |
| Combination | 2.7 ± 0.49 | 0.58 ± 0.36 | 1.44 ± 0.09 | 0.17 ± 0.01 | 0.36 ± 0.01 | 0.17 ± 0.02 |
| KMUP₅ | 1.58 ± 2.24 | 0.14 ± 0.39 | 1.32 ± 1.59 | 0.06 ± 0.11 | 0.05 ± 0.3 | 0.01 ± 0.16 |
| CAM | 3.23 ± 0.52 | 0.68 ± 0.57 | 1.64 ± 1.49 | 0.16 ± 0.16 | 0.5 ± 0.33 | 0.25 ± 0.16 |

WBC total (white blood cell total count), NEU(neutrophil), LY(lymphocyte), MO(monocyte), EOS (eosinophil), BA (basophil)
CTL(control), BLM (Bleomycin), Sim (Simvastatin), Combination (Simvastatin 2.5 mg/kg in combination wih KMUP-1 2.5 mg/kg), KMUP₅ (KMUP-1-Simvastatinic Acid), CAM (Clarithromycin)
n = 6
#: p < 0.05 compared with CTL group;
* p < 0.05,
** p < 0.01 compared with BLM group.

IV. Collagen Aggregation

The amount of the collagen in the lung lavage is measured to reflect the fibrosis formation. Table 3 shows the collagen amount in the aveolar lavage of mice administrated with bleomycin on day 28, the results of the fibrosis formation as shown in FIG. 6 are correspondent with the result in Masson trichrome, which illustrates that the KMUP-1 and KMUP-5 administration may reduce the fibrosis formation in lung tissue (p<0.01).

TABLE 3
the inflammatory cells in the alveolar lavage of mice
administrated with BLM on day 28

| | The amount of collagen (μg/lung) |
|---|---|
| CTL | 128.86 ± 0.04 |
| BLM | 191.90 ± 0.04^{##} |
| KMUP₁-1 mg | 165.16 ± 0.11 |
| KMUP₁-2.5 mg | 132.25 ± 5.95** |
| KMUP₁-5 mg | 108.32 ± 6.05** |
| Sim | 140.53 ± 0.05* |
| Combination | 96.71 ± 0.03* |
| KMUP₅ | 79.40 ± 0.02** |
| CAM | 86.07 ± 0.02* |

CTL (control),
BLM (Bleomycin),
Sim (Simvastatin),
Combination (Simvastatin 2.5 mg/kg in combination with KMUP-1 2.5 mg/kg),
KMUP₅ (KMUP-1-Simvastatinic Acid),
CAM (Clarithromycin)

V. The Inhibitory Effects of KMUP-1 and KMUP-5 on MMP-2 and MMP-9

1. Lung Lavage

The activities of MMP-2 (FIGS. 7A and 8A) and MMP-9 (FIGS. 7B and 8B) in the lung lavage of mice on day 7 (FIG. 7) and day 28 (FIG. 8) are analyzed by zymography. The expression levels are minor in the normal control group. The bleomycin administration may significantly increase the MMP-2 and MMP-9 expressions whatever on day 7 or day 28. The expressions of MMP-2 and MMP-9 on day 7 are higher than that on day 28. The KMUP-1 and KMUP-5 administrations may significantly decrease the MMP expression whatever on day 7 or day 28 (p<0.05). It is seen that KMUPs have the effect of mitigating the inflammation.

2. Lung Tissue

The lung tissue is analyzed by western blotting, and the MMP-2 and MMP-9 protein expressions are shown in both of the FIGS. 9 and 10. In the lung tissue, the MMP-2 expression is more significant than MMP-9. KMUP-1 and KMUP-5 may mitigate the expressions of MMP-2 and MMP-9 increased by administrating the bleomycin (p<0.01).

VI. KMUP-1 and KMUP-5 Affect the Protein Expression

1. KMUP-1 and KMUP-5 Affect the HMGB1 Protein Expression

FIG. 11 shows the HMGB1 protein expression in the lung tissue analyzed by western blotting.

2. KMUP-1 and KMUP-5 Affect the TGF-β, CTGF, Smad3 and p-Smad3 Protein Expressions

The TGF-β (FIG. 12), CTGF (FIG. 13) and Smad3/p-Smad3 (FIG. 14) protein expressions in the lung tissue are analyzed by western blotting.

3. KMUP-1 and KMUP-5 Affect the eNOS, phosphor-eNOS (p-eNOS) and iNOS Protein Expressions

The eNOS (FIG. 15), p-eNOS (FIG. 16) and iNOS (FIG. 17) protein expressions in the lung tissue are analyzed by western blotting. It is found that the eNOS expression is more associated with lung fibrosis than iNOS in the pathology group. The KMUPs administration effectively increases the eNOS expression.

4. KMUP-1 and KMUP-5 Affect the RhoA Protein expression

The RhoA protein expression in the lung tissue is analyzed by western blotting as shown in FIG. 18.

Other objects, advantages and efficacies of the present invention will be described in detail below taken from the preferred embodiments with reference to the accompanying drawings, in which:

### BRIEF DESCRIPTION OF THE DRAWINGS

FIG. 1 shows the structures of the KMUP-1.

FIG. 2 shows the structures of the KMUP-5.

FIG. 3 shows the experimental protocol of the IPF animal model.

FIG. 4 shows the total cell count in bronchoalveolar lavage fluid (BALF) on day 7(A) and day 28 (B) after BLM injection (n=6).

FIG. 5 shows the number of neutrophill, lymphocyte, monocyte, eosinophill and basophill in BALF on day 7 (A) and day 28 (B) after BLM injection (n=6).

FIG. 6 shows the collagen content in lung tissue on day 28 after BLM injection (n=6).

FIG. 7 shows the gelatin zymography of MMP-2 (A) and MMP-9 (B) in BALF on day 7 after BLM injection (n=6).

FIG. 8 shows the gelatin zymography of MMP-2 (A) and MMP-9 (B) in BALF on day 28 after BLM injection (n=6).

FIG. 9 shows the MMP-2 (A) and MMP-9 (B) expressions in lung by western blotting on day 7 after BLM injection (n=6).

FIG. 10 shows the MMP-2 (A) and MMP-9 (B) expressions in lung by western blotting on day 28 after BLM injection (n=6).

FIG. 11 shows the HMGB1 expression in lung by western blotting on day 7 (A) and Day 28 (B) after BLM injection (n=6).

FIG. 12 shows the TGF-beta expression in lung by western blotting on day 7 (A) and day 28 (B) after BLM injection (n=6).

FIG. 13 shows the CTGF expression in lung by western blotting on day 7 (A) and day 28 (B) after BLM injection (n=6).

FIG. 14 shows the Smad3/p-Smad3 expression in lung by western blotting on day 7 (A) and day 28 (B) after BLM injection (n=6).

FIG. 15 shows the eNOS expression in lung by western blotting on day 7 (A) and day 28 (B) after BLM injection (n=6).

FIG. 16 shows the p-eNOS expression in lung by western blotting on day 7 (A) and day 28 (B) after BLM injection (n=6).

FIG. 17 shows the iNOS expression in lung by western blotting on day 7 (A) and day 28 (B) after BLM injection (n=6).

FIG. 18 shows the RhoA expression in lung by western blotting on day 7 (A) and day 28 (B) after BLM injection (n=6).

FIG. 19 shows the ET1 expression in lung by western blotting on day 7 (A) and day 28 (B) after BLM injection (n=6).

FIG. 20 (A) (B) shows the proposed mechanisms of action of KMUP-1/KMUP-5 on mice lung tissue.

FIG. 21 shows the experimental protocol of the OVA-sensitized and -challenged animal model, wherein OVA is intraperitoneally injected on days 1 and 8, OVA (1%) or OVA (1%)+KMUP-1 was nebulized into mice's airway on days 21˜27 and mice are sacrificed on day 28.

FIG. 22 shows the effects of short-term inhalation of KMUP-1 (A) and L-NAME-pretreatment (B) on eNOS expression in lung tissues.

FIG. 23 shows the effects of short-term inhalation of KMUP-1 (A) and L-NAME-pretreatment (B) on MMP-9 expression in lung tissues.

FIG. 24 shows the effects of KMUP-1 on eNOS (A) and iNOS (B) protein expressions in lung tissue of OVA-sensitized and OVA-challenged mice.

FIG. 25 shows the effects of KMUP-1 on sGCα1 (A) and PKG (B) protein expressions in lung tissue of OVA-sensitized and OVA-challenged mice.

FIG. 26 shows the effects of KMUP-1 on MMP-9 protein expression in lung tissues of OVA sensitized and OVA-challenged mice, determined by western blotting (A) and gelatin zymography (B).

FIG. 27 shows the effects of KMUP-1 on ICAM-1 (A) and VCAM-1 (B) protein expression in lung tissues of OVA sensitized and OVA-challenged mice, determined by western blotting.

FIG. 28 shows the effect of KMUP-1 on the vascular (A) and bronchial (B) wall thickness of OVA-sensitized and OVA-challenged mice.

FIG. 29 shows the effects of KMUP-1 on NOx levels (A) and cellular components (B) in BALF.

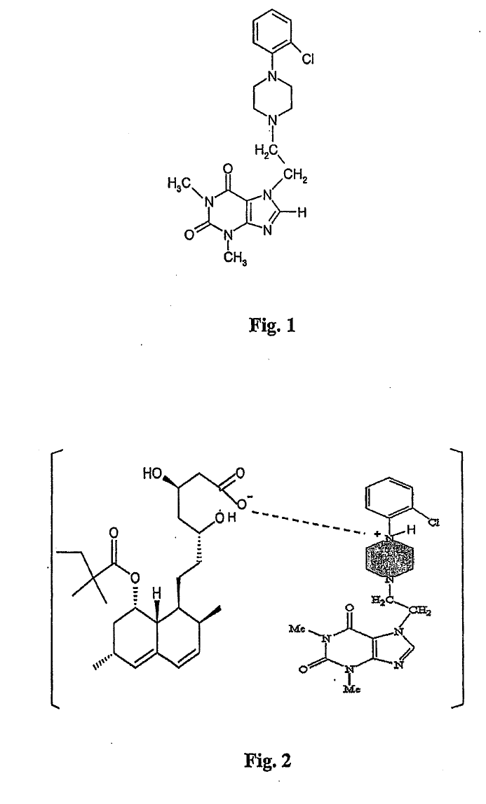

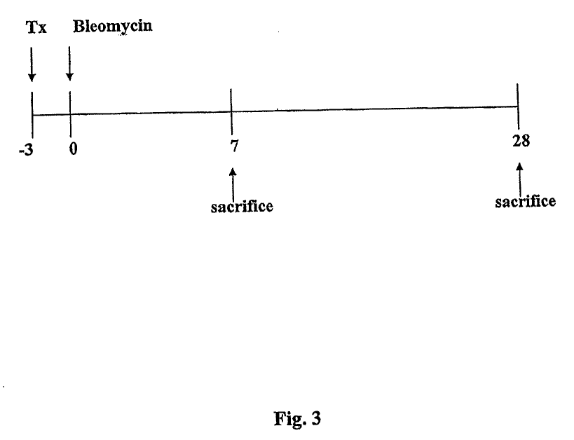

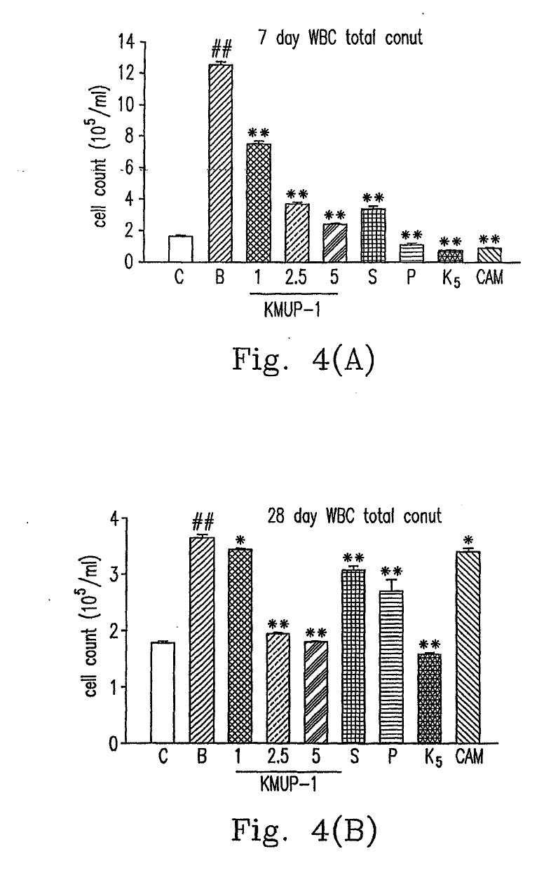

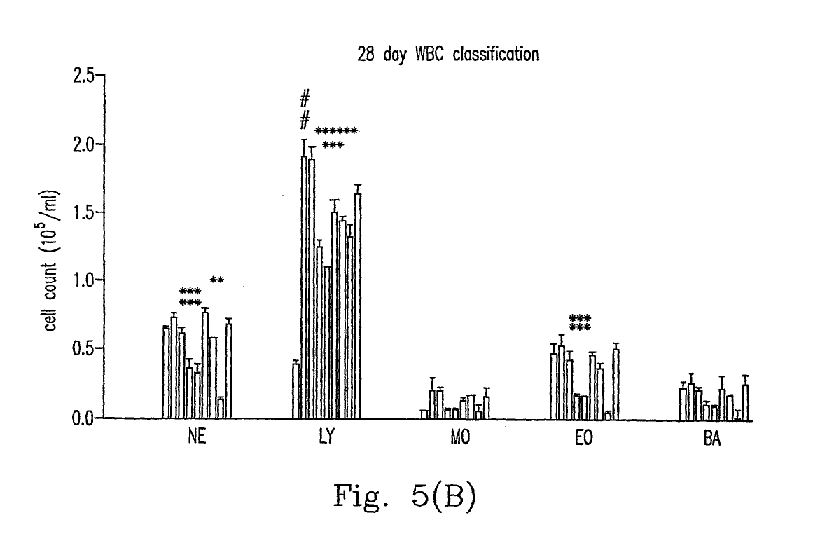

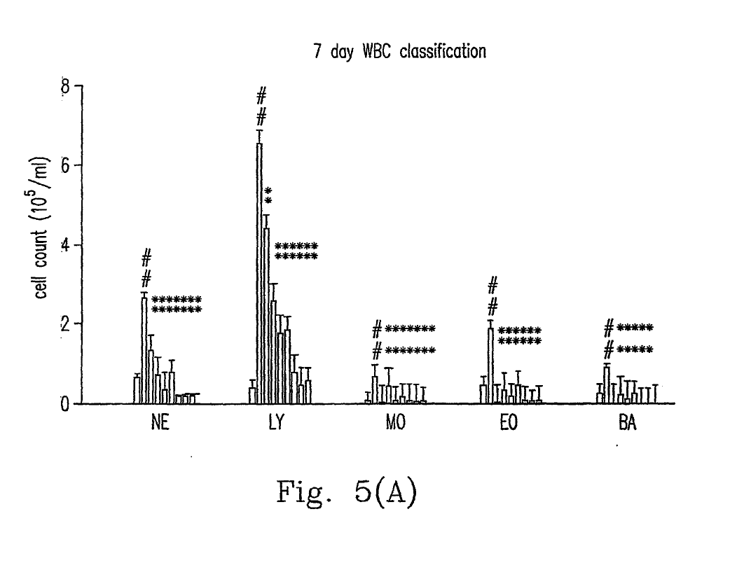

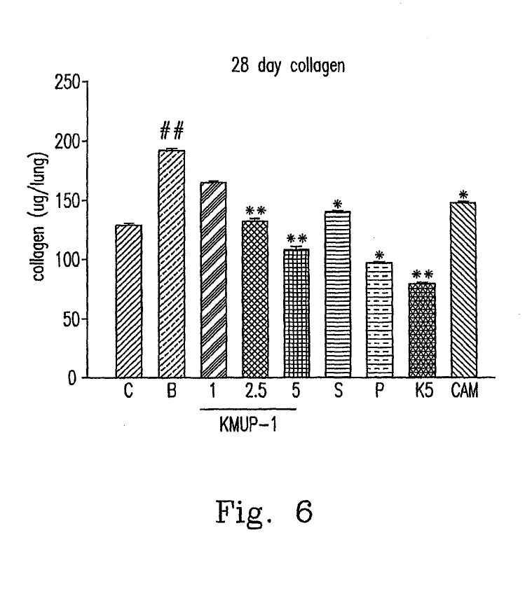

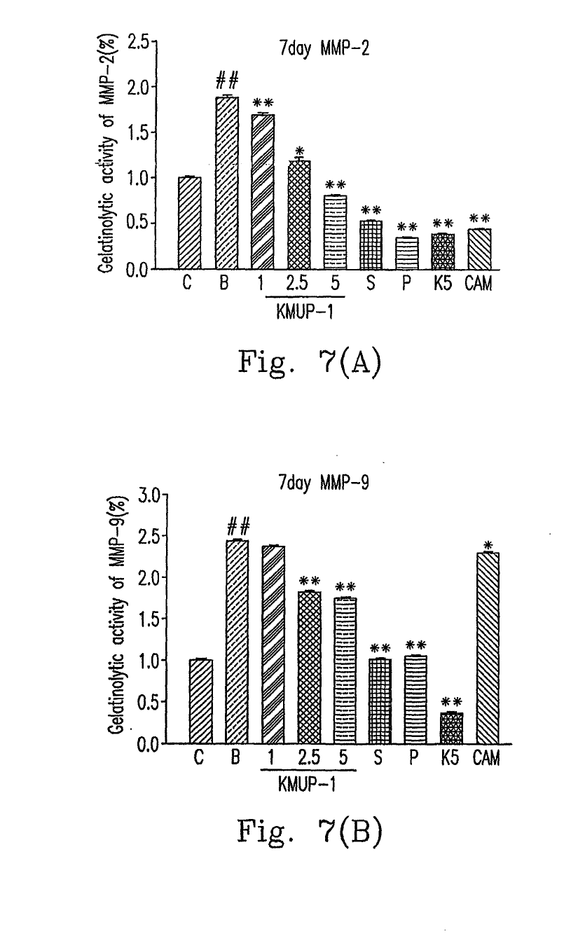

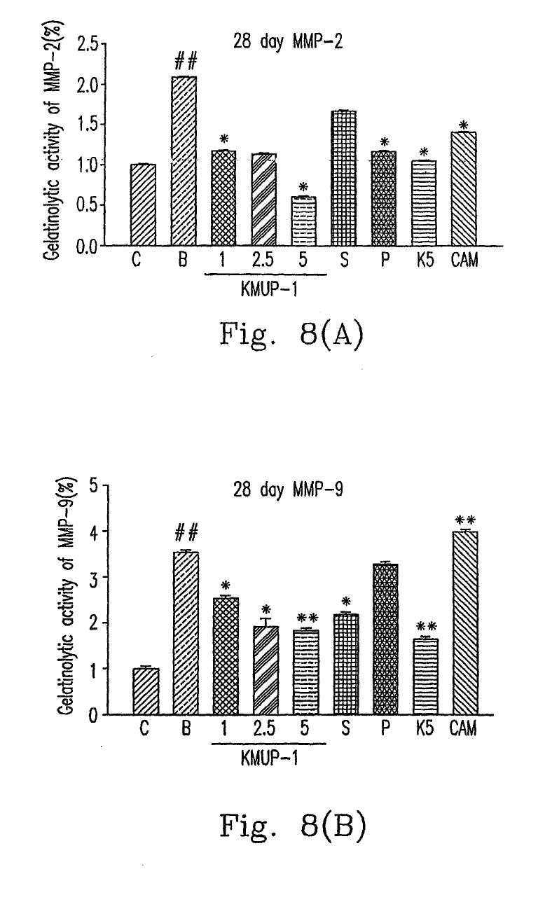

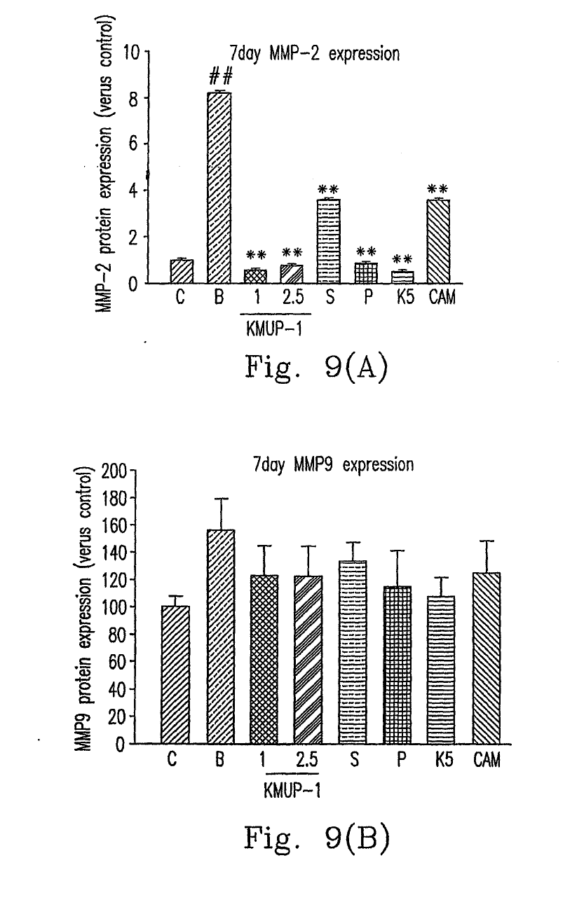

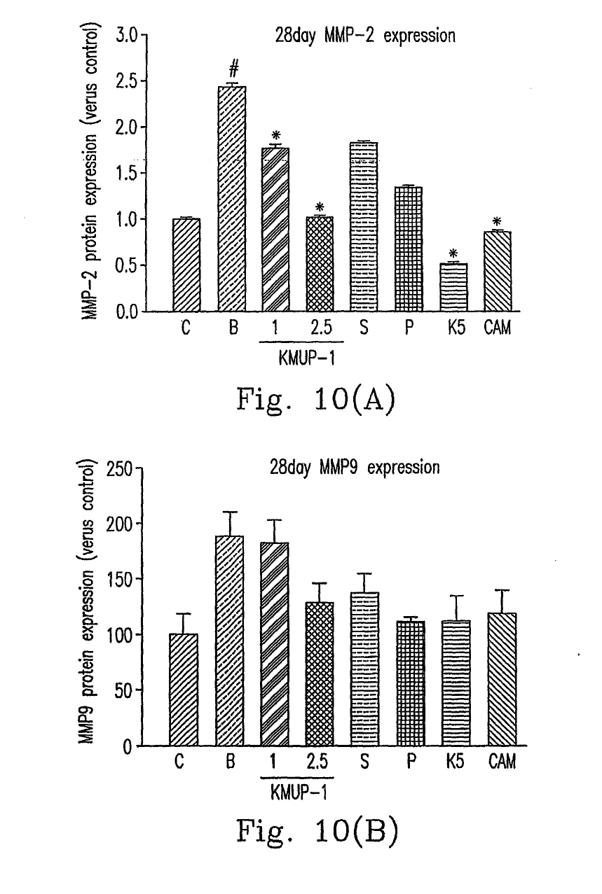

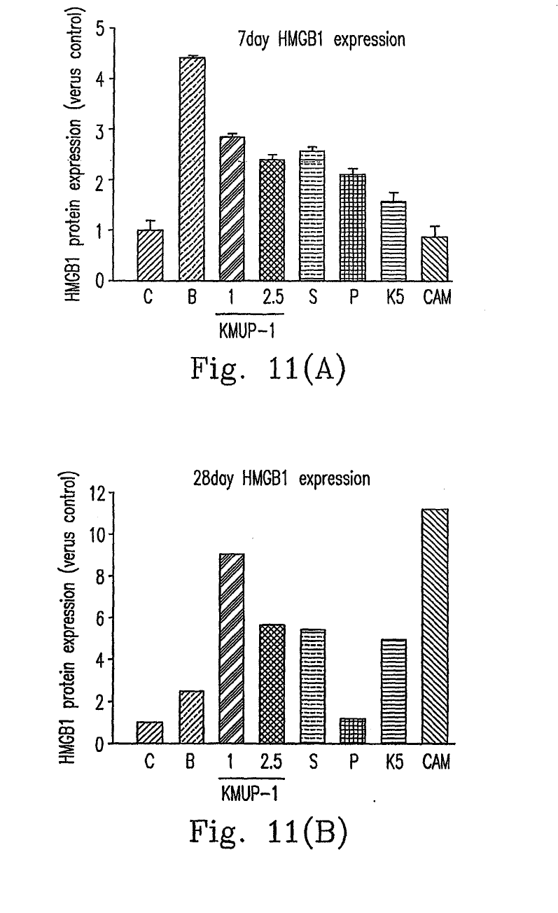

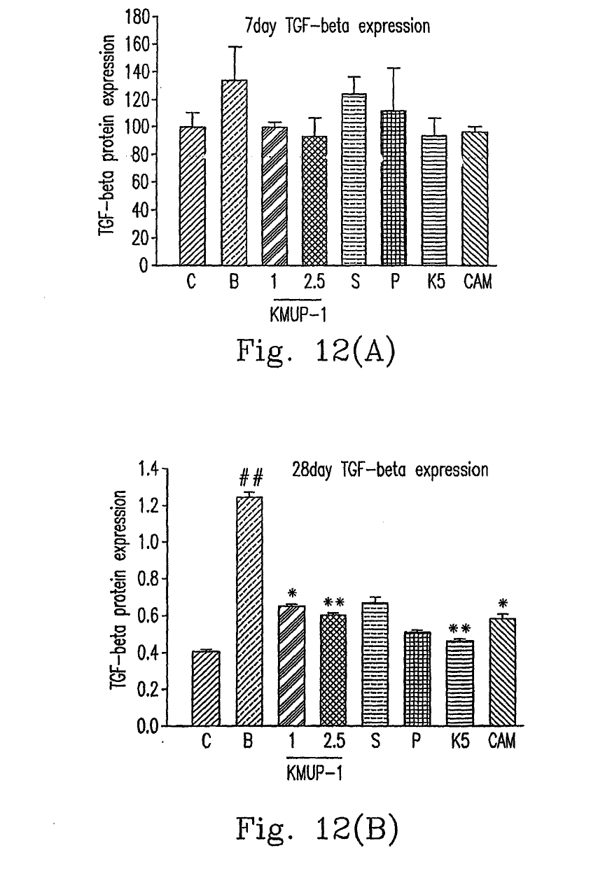

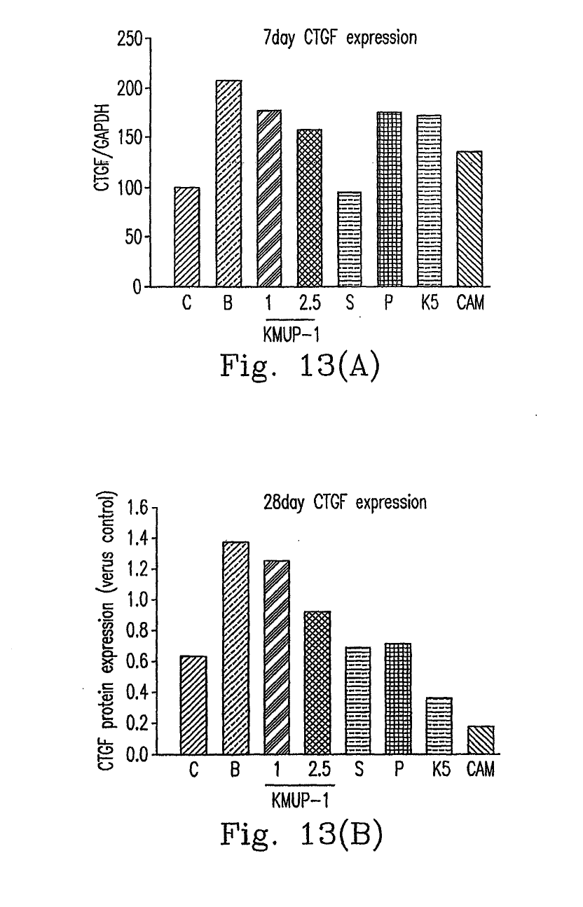

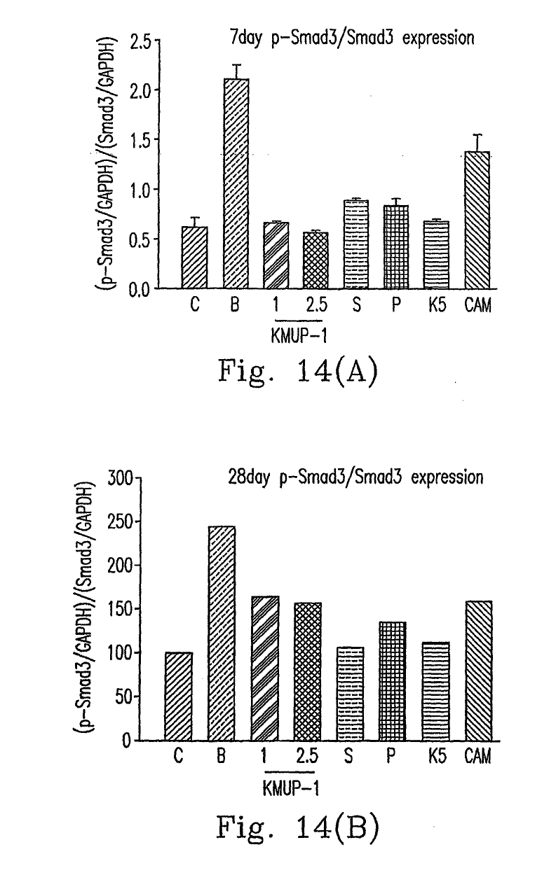

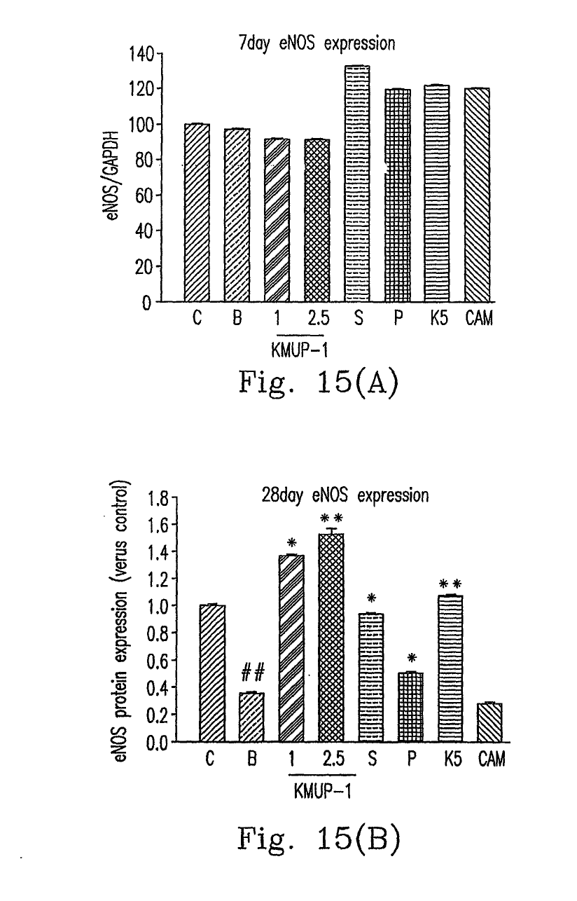

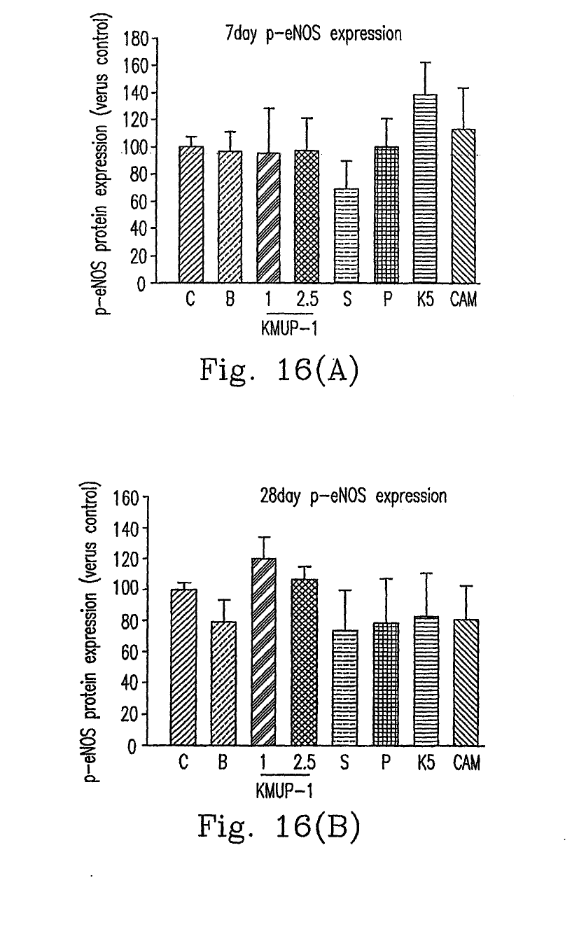

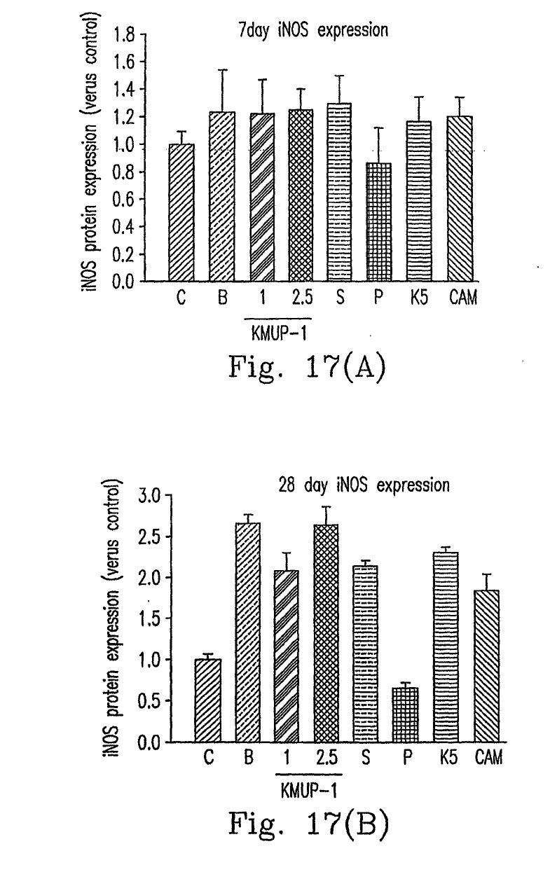

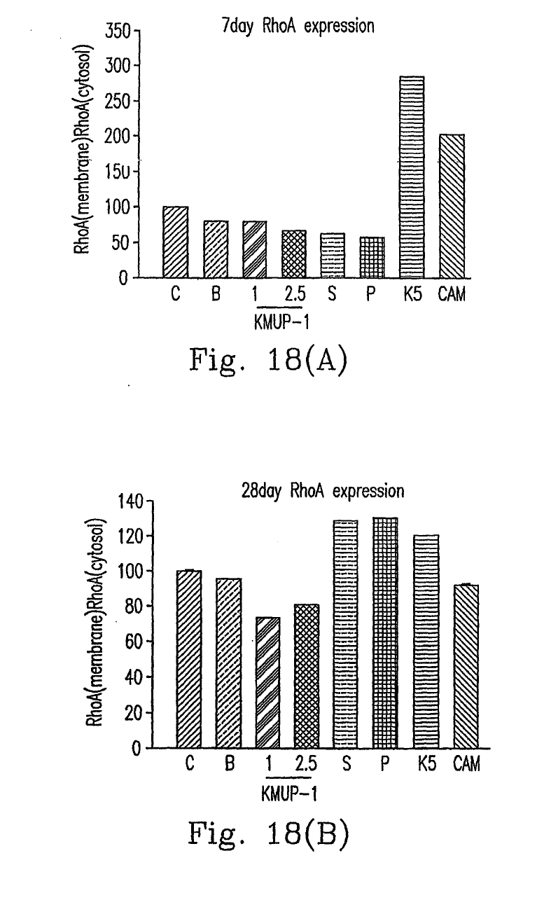

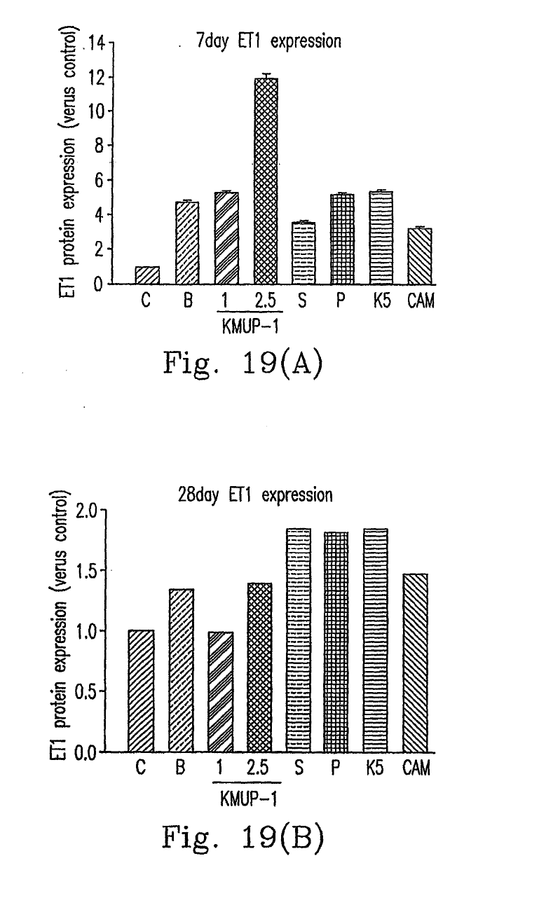

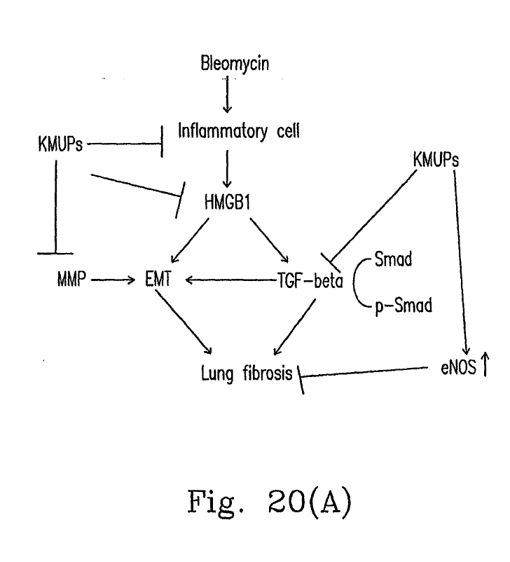

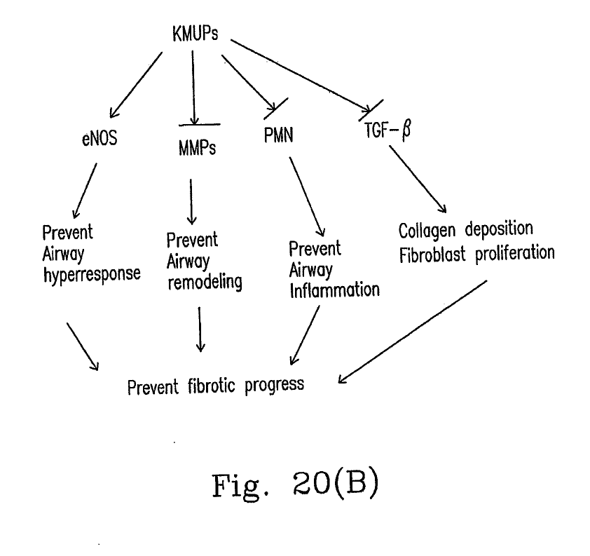

### DETAILED DESCRIPTION OF THE PREFERRED EMBODIMENT

The relative biochemical experiments are performed by the way described below.

(I) The Preparation of Each Kind of Agent

### Western Blot Analysis

A. 1.5M Tris(hydroxymethyl)aminomethane HCl (Tris-HCl) buffer solution at pH 8.8:27.23 g Tris base dissolving in 80 ml distilled water is adjusted with 1N HCl to pH 8.8, finally added by the distilled water to make the final volume as 150 ml, and stored at 4° C.

B. 0.5 M Tris-HCl at pH 6.8:6.0 g Tris base dissolving in 60 ml bidistilled water is adjusted with 1N HCl to pH 6.8, finally added by the bidistilled water to make the final volume as 100 ml, and stored at 4° C.

C. 10% sodium dodecyl sulfate (SDS): 10 g SDS is gently mixed in 90 ml distilled water, finally added by the distilled water to make the final volume as 100 ml, and stored under room temperature.

D. The sample buffer (containing the reducing buffer of SDS): 1.0 ml 0.5M Tris-HCl at pH 6.8, 0.8 ml glycerol, 1.6 ml 10% (w/v) SDS, 0.4 ml 2-mercaptoethanol and 1% (w/v) bromophenolblue are respectively taken for storing at room temperature.

E. 5× electron buffer: 9.0 g Tris base, 43.2 g glycerol and 3.0 g SDS are respectively weighted and adjusted to pH 8.3, the distilled water is added therein to 600 ml for storing at 4° C. The 5× electron buffer is diluted as 1× electron buffer by using the distilled water before use.

F. Transfer buffer: 3.025 g Tris base (25 mM) and 14.413 g glycine (192 mM) are respectively weighted and added by 200 ml methanol (20%) and 800 ml distilled water to make the final volume as 1 L.

G. T-TBS: 2.42 g Tris base and 8.0 g sodium chloride are respectively weighted and adjusted as pH 7.6, added by 1.0 ml polyoxyethylene sorbitan mono-laurate (Tween-20) and further added by 900 ml distilled water to make the final volume as 1 L.

H. Blocking buffer: 5.0 g skim milk is dissolved in 100 ml T-TBS.

I. Stripping buffer at pH 7.6:9.85 g Tris-HCl and 2.0 g SDS are weighted respectively, added by 6.8 ml 2-mercaptoethanol and finally added by the distilled water to make the final volume as 1 L. The use: it is used for washing away the antibody measured at the first time to measure the activity of another antibody.

J. The preparation of the upper and the lower gels

Separating gel, the lower gel-0.375 M Tris-HCl at pH 8.8

| | 12% | | 7.5% | | 10% | | | |
|---|---|---|---|---|---|---|---|---|
| | (50 KD↓) | | (60 KD↑) | | (10-100 KD) | | | |
| | 2 gels | | 2 gels | | 2 gels | | 4 gels | |
| bidistilled water (DDW) | 3.35 | mL | 4.85 | mL | 4.01 | mL | 8.02 | mL |
| 1.5M Tris-HCl at pH 8.8 | 2.5 | mL | 2.5 | mL | 2.5 | mL | 5 | mL |
| 10% (w/v) SDS stock | 100 | μL | 100 | μL | 100 | μL | 200 | μL |
| (room temperature) | | | | | | | | |
| Acr/Bis (30% stock) | 4 | mL | 2.5 | mL | 3.34 | mL | 6.68 | mL |
| APS (10%) | 50 | μL | 50 | μL | 50 | μL | 100 | μL |
| TEMED | 5 | μL | 5 | μL | 5 | μL | 10 | μL |
| Total volume | 10 | mL | 10 | mL | 10 | mL | 20 | mL |

Acr/Bis: Acrylamide/Bis-Acrylamide
APS: amminonium persulfate
TEMED: N,N,N′,N′-Tetramethylethylenediamine

Stocking gel, the upper gel-4% 0.125 M Tris-HCl at pH 6.8

| | 4 gels | | 2 gels | |
|---|---|---|---|---|
| DDW | 6.1 | mL | 3.05 | mL |
| 0.5M Tris-HCl at pH 6.8 | 2.5 | mL | 1.25 | mL |
| 10% (w/v) SDS stock | 100 | μL | 50 | μL |
| Acr/Bis (30% stock) | 1.33 | mL | 0.65 | mL |
| APS (10%) | 50 | μL | 25 | μL |
| TEMED | 10 | μL | 5 | μL |
| Total volume | 10 | mL | 5 | mL |

K. Antibody dilution:

| | | | Secondary |
|---|---|---|---|
| Ab | Primary antibody | KDa | antibody |
| eNOS | 1:500 | 135 | R |
| p-NOS | 1:500 | 135 | M |
| iNOS | 1:500 | 135 | M |
| MMP-9 | 1:1000 | 92 | Gout |
| MMP-2 | 1:200 | 62-72 | R |
| Smad3 | 1:1000 | 48 | R |
| p-Smad3 | 1:1000 | 48 | R |
| CTGF | 1:10000 | 42-44 | R |
| β-actin | 1:20000 | 43 | M |
| GAPDH | 1:40000 | 38 | M |
| HMGB1 | 1:1000 | 33 | R |
| TGF-β | 1:500 | 25 | M |
| RhoA | 1:1000 | 22 | M |
| ET1 | 1:1000 | 24 | G |

GAPDH: Glyceraldehyde 3-phosphate dehydrogenase
R (Rabbit),
M (Mouse),
G (Goat)

(2) Zymography

A

Triton-X 100 buffer

| | |
|---|---|
| 1 M Tris-HCl, pH 7.5 | 25 ml |
| d.d. H₂O | 462.5 ml |
| Triton-X100 | 12.5 ml |

d.d. H₂O (bidistilled water)

B

Developing buffer

| | | |
|---|---|---|
| CaCl₂ | 0.6059 | g |
| NaCl | 4.47066 | g |
| 1M Tris-HCl, pH 7.5 | 25 | mL |
| d.d.H₂O | 475 | ml |

C

Stain buffer

| | |
|---|---|
| Comassie blue R-250 | 1 g |
| Acetic acid | 50 ml |
| Methanol | 225 mL |
| d.d. H₂O | 225 ml |

D

Destain buffer

| | |
|---|---|
| Acetic acid | 75 ml |
| Methanol | 50 mL |
| d.d. H₂O | 375 ml |

E

Non-reduced Sample buffer

| | | |
|---|---|---|
| d.d. H₂O | 44.76 | mL |
| 0.5 M Tris-HCl, pH 6.8 | 30.24 | mL |
| Glycerol | 25 | mL |
| SDS | 5 | g |
| Bromophenol Blue | 2 | mg |

The non-reduced sample buffer is prepared as 5x stock for storing at room temperature.

F. 1.5 M Tris-HCl at pH 8.8:27.23 g Tris base dissolving in 80 ml distilled water is adjusted with 1N HCl to pH 8.8, finally added by the distilled water to make the final volume as 150 ml, and stored at 4° C.

G. 0.5 M Tris-HCl at pH 6.8:6.0 g Tris base dissolving in 60 ml distilled water is adjusted with 1N HCl to pH 6.8, finally added by the distilled water to make the final volume as 100 ml, and stored at 4° C.

H. 10% SDS: 10 g SDS is gently mixed in 90 ml distilled water, finally added by the distilled water to make the final volume as 100 ml, and stored under room temperature.

I. 1% gelatin: 10 mg gelatin is sampled and gently mixed in 0.5 ml distilled water, and finally added by the distilled water to make the final volume as 1 ml before use.

J.

Running gel preparation:

| | |
|---|---|
| d.d. H₂O | 3.37 mL |
| 1.5M Tris-HCl, pH 8.8 | 2.5 mL |
| 1% gelatin | 1 ml |
| 10% SDS | 0.1 ml |
| Acr/Bis (30% stock) | 2.5 mL |
| 10% APS | 0.05 ml |
| TEMED | 6.25 μL |

Stacking gel preparation:

| | |
|---|---|
| d.d. H₂O | 2.95 mL |
| 0.5M Tris-HCl, pH 6.8 | 0.5 mL |
| 10% SDS | 0.04 ml |
| Acr/Bis (30% stock) | 0.5 mL |
| 10% APS | 0.04 ml |
| TEMED | 4 μL |

*After being mixing evenly’ the stacking gel is added on the seperating gel and inserted the comb to form the wells.

### APS (Ammonium Persulfate)

(II) The Studying Methods

1. Intratracheal Administration

After being anesthed with pentobarbital in the dose of 0.52 mg/10 g, the mice is kept upright and positioned an otoscope into the larynx for looking into the trachea clearly. The bleomycin is loaded by a loading tip for intratracheal administration.

2. Sample Collection

(1) Bronchoalveolar Lavage Fluid (BALF)

The mice are euthanatized by administering the Urathane in a dose of 37.5 mg/10 g. The larynx is opened, the trachea is picked out, cut and held by a forcepts and a 0.58 mm PE tube is slowly inserted the trachea for infusing 0.5 ml PBS. The PBS is incubated for 15 seconds and then collected after gently massaging the lung tissue. 1 c.c. lung lavage is obtained after the above steps are repeated twice. The lung lavage is collected and transferred into a centrifuged tube for centrifuging at 1,200 rpm at 4° C. for 6 mins. The supernatant is collected and stored at −80° C. for zymography, and the pellet is suspended in PBS for calculating the WBC count.

(2) After the lung lavage is obtained, the chest of mice is opened and the lung lobes are cut one by one. The left lung is immersed in 10% formalin for tissue histology, and the right lung is use for protein analysis and collagen amount measurement.

3. Analysis for the Inflammatory Cell Amount and Kind

The pellet of the alveolar lavage is suspended in PBS and transferred into an eppendorff. The amount and kind of the inflammatory cell such as WBC total, the lymphocyte, the monocyte, the neutrophil, the eosinophil and the basophil are analyzed by an automatic animal blood cell analyzer.

4. Histology Analysis

The with 10% formalin treated and freezed lung is placed in a tissue dehydrating machine, and the tissue is fixed, dehydrated, washed and rinsed in turn. The tissue is further prepared as a wax-embedded tissue by a tissue embedded machine through appropriate cooling. The wax-embedded tissue is cut by a sliding microtome. The slices about 4 μm are placed on the slides and extended in 35° C.-50° C. warm water, and then dried in a 56° C.-60° C. extender for staining

(1) Hematoxylin-Eosin Stain (H&E Stain)

The slices are successively dehydrated via 50%-99.9% ethanol, incubated in xylene for 2 hrs and the incubation step is repeated twice. The dehydration and wax infiltration are completed through infiltrating in 58° C. wax for 2 hrs and repeating the process twice. The wax-embedded tissue is cut by a sliding microtome as 4 μm sample and double stained with hematoxylin and eosin.

After incubating in xylene, 99.9%-70% ethanol and RO water, the slices are stained with hematoxylin and eosin, respectively. Finally, the slices are incubated in 70%-99.9% ethanol, mounted for observating the pathological change under the microscope.

(2) Masson's Trichrome Stain

The dehydrated and wax infiltrated slides are reacted with Bouin's solution for 60 mins, after reaching 60° C., the slides are washed with RO water for 5 mins. After being rinsed, the slides are reacted with Weigert's solution for 10 mins, with soluble Biebrich scarlet for 5 mins and with phosphomolybdic acid solution for 10 mins. Then, the slides are reacted with aniline blue for 5 mins, with 1% acetic acid for 1 min, dried for 2 min and covered by the cover slides for completing the staining. After the staining, the nuclear is black, the cytosol, muscle and red blood cell are red and the collagen is blue.

5. Bio-Rad DC Protein Assay

The amount of protein is measured by the protein stock and silver staining agent (Bio-Rad). The silver staining agent is an acidic solution containing the commassie blue, and its absorption wavelength is shifted fro 465 nm to 595 nm when it binds to a protein and forms a complex. Depending on this characteristic, the absorption at 595 nm is directly measured by an enzyme immunoassay analyzer (ELISA reader), and the mass of the protein to be tested can be obtained by referring to the standard curve of the standard solution.

0.1 mg ml⁻¹ bovine serum albumin (BSA) is taken as a protein standard and prepared as known protein standard solutions such as 0, 2, 4, 8, 12, 16, 20 an 30 μg ml⁻¹.

The concentration of the protein lysate is measured in 237 μl bidistilled water supplemented with 3 μl cell lysate and each of 60 μl protein stock for color reaction. The absorption at 595 nm wavelength is measured by the ELISA reader and the concentration of the protein lysate is speculated by referring to the standard curve.

6. Western Blotting

(1) The mice lung is immersed in the tissue lysis buffer and homogenized by an ultrasound sonicator on ice.

(2) The lysate is centrifuged at 13,000 g under 4° C. for 30 mins. The supernatant is collected and the protein concentration measurement adopts the“Bio-Rad DC protein assay”. After diluting the protein as specific concentrations, the sample buffer being one fourth of the sample volume is added thereinto for heating at 100° C. for 5 mins, and the sample is loaded into each wells of SDS-PAGE gel in turn. The running buffer is added into the electrophoresis tank for electrophoresis at 100 V, the voltage is adjusted as 200 V when the protein runs to the lower gel, and the electrophoresis is stopped when the SDS-PAGE dye enters the SDS-PAGE.

(3) The PVDF membrane is rinsed ith transfer buffer for use, and the protein on the SDS-PAGE is transferred to the PVDF membrane. The SDS-PAGE layer covering on the PVDF membrane is placed in a protein transfer tank immersed with the transfer buffer with the current set at 100V. After 80 mins, the transfer is completed and then the PVDF membrane is taken out.

(4) after cutting the band having an appropriate molecular weight on the PVDF membrane, an appropriate amount of the blocking buffer is added to for blocking under room temperature to remove the non-specific binding. Then, evenly covering the diluted primary antibody on the PVDF membrane to react under room temperature for 2 hrs, and the excessive antibodies are removed by T-TBS. Further, evenly covering the diluted secondary antibody to react under room temperature for 1 hr and finally washing the excessive antibodies with T-TBS. The enhanced chemiluminescence (ECL) agent is added for reacting for 1-90 secs. After exposing to a x-ray film, the reaction is completed.

7. Zymography

The MMPs in the lung lavaga are measured by zymography. It is known that all of the MMP-1, MMP-2, MMP-3, MMP-7 and MMP-9 can degrade the gelatin, especially the MMP-2 and MMP-9. The activity of collagenase can be analyzed by SDS-PAGE gel adding by gelatin. After the collected bronchoalveolar lavage fluid (BALF) is electrophoreted, the SDS is removed with 2.5% (v/v) Triton X-100 and the activity of collagenase is recovered to degrade the gelatin. After reacting for a period of time, the commassie blue is utilized for staining and destaining so that a transparent band in the area having the collagenase activity can be seen owing to the gelatin is degraded.

(1) BALF Collection and Quantitation

The BALF is collected and the concentration thereof is measured by Bio-Rad DC protein assay. The absorption of BALF at 595 nm wavelength is measured by an ELISA reader with a BSA standard, and the concentration of the sample is measured according to the standard curve of this absorption value.

(2) Electrophoresis Analysis and Reaction

The sample is mixed with non sample buffer. After reacting under temperature for 10 mins, the separating gel containing 0.1% gelatin is utilized for electrophoresis analysis under low temperature. While the electrophoresis is finished, the gel is immersed in 2.5% Triton X-100 buffer to react under temperature for half hour. The 2.5% Triton X-100 buffer is exchanged to react under temperature for additional half hour. The gel is immersed in the developing buffer at 37° C. for 24 hrs, stained with staining buffer for 1 hr and destained with the destaining buffer, and the transparent band can be seen.

8. Collagen Analysis of The Lung Tissue

The 1.5 ml eppendorffs are taken to arrange the calibration curve as follows.

| Relative collagen (μg) | 0 | 5 | 10 | 25 | 50 |
|---|---|---|---|---|---|
| Standard collagen | 0 μl | 10 μl | 20 μl | 50 μl | 100 μl |
| distilled water | 100 μl | 90 μl | 80 μl | 50 μl | 0 μl |

Sample preparation: 20 μl sample is dissolved in 80 μl distilled water. 1 ml dye is added into the eppendorff for vortexing for 1 min, and the eppendorff is placed on a shaker for 35 mins. The eppendorff is then centrifuged at 12,000 rpm for 10 mins. The supernatant in the eppendorff is poured out slowly, 1 ml basic compound is added for dissolving the precipitation. After being violently shaken for 5 mins, the reaction is incubated for 3 hrs to stabilize the color.

0.2 ml sample is taken from each eppendorff and loaded into a 96 well plate. The amount of collagen is estimated through the 540 nm absorption measured by the ELISA reader.

9. Inhibition for the Asthma in the Animal

The male BALB/c mice (20-23 g) are injected (i.p.) with OVA (5 mM, 30 mins), sensitized with OVA on day 1 and day 8 via i.p injection and activated by 2 mg aluminum hydroxide to which 2 μg OVA is absorbed. The spray of 5 mM/30 mins/day KMUP-1 HCl salt or the related complex salt is administered on days 21 to 27. The asthmatic animals are divided into 5 groups, wherein the control group is only administered with saline spray and the remaining experimental groups are firstly administered with 1% OVA spray and then administered with the sprays of the related complex salts respectively as shown in Table 4. The inhibition of the expression of OVA-induced MMP-9 in lung tissue of BALB/c mice is measured.

TABLE 4
Inhibition for the asthma in the animal

| Drug 5 mM | Inhibition % of the MMP-9 |
|---|---|
| OVA + Vehicle Control | 100% |
| OVA + KMUP-1 HCl salt | 55% |
| OVA + KMUP-1-Cromolyn | 60% |
| OVA + KMUP-1-Nedocromil | 63% |
| OVA + KMUP-1-Montelukast | 70% |
| OVA + KMUP-1-Acetylcysteine | 68% |
| OVA + KMUP-1-Arginine | 65% |

10. The Monocrotaline (MCT)-Treated Rats Show the Decreased Pulmonary Aterial Hypertension

The rats administered MCT (60 mg/kg) are caused pulmonary artery hypertension on day 21 after the intraperitoneal injection. The KMUP-1HCl salt (2.5 mg/kg), Simvastatin (2.5 mg/kg), KMUP-1-nicotinic acid complex (2.5 mg/kg), KMUP-1-citric acid complex (2.5 mg/kg), KMUP-1-Simvastatinic Acid complex (2.5 mg/kg) and KMUP-1-γ-polyglutamic acid complex (2.5 mg/kg) orally administered, or the KMUP-1-PGI₂ complex (0.1 mM) inhaled every day may inhibit the pulmonary arterial blood pressure (PABP) in MCT-treated male Wistar rats.

TABLE 5
The inhibition of pulmonary hypertension

| | | PABP |
|---|---|---|
| Group | Dose (mg/kg/day) | (mmHg) |
| Control group | | 11 ± 1.4 |
| MCT (60 mg, s.c. injection) | | 25 ± 2.3 |
| MCT + KMUP-1 HCl salt | 2.5 mg, p.o. | 13 ± 1.7 |
| MCT + KMUP-1-Citric acid | 2.5 mg, p.o. | 16 ± 1.8 |
| MCT + KMUP-1-Nicotinic acid | 2.5 mg, p.o. | 12 ± 2.6 |
| MCT + KMUP-1-Simvastatinic acid | 2.5 mg, p.o. | 12 ± 1.3 |
| MCT + KMUP-1-Glycerrhizic acid | 2.5 mg | 10 ± 1.2 |
| MCT + KMUP-1-γ- | 2.5 mg, p.o. | 11 ± 0.6 |
| Polyglutamic Acid | | |
| MCT + KMUP-1-PGI₂ | 0.1 mM, (inhalation) | 10 ± 0.2 |
| MCT + KMUP-1-GLT Co-polymer | 2.5 mg | 11 ± 0.2 |
| acid | | |

11. KMUP-1 Salts Inhibit the Lung Fibrosis in Mice Induced by Bleomycin (BM) Mediated Via Transforming Growth Factor Beta (TGF-β) Expression in Lung

The increased amount of the collagen synthesis and the macrophage generation in alveolus may facilitate the formulation of TGF-β, and the expression of TGF-β is deeming as a biomarker of the lung fibrosis. KMUP-1HCl salt (1, 2.5 and 5 mg/kg), the Simvastatin (5 mg/kg), the KMUP-1-nicotinic acid complex (2.5 mg/kg), the KMUP-1-Simvastatinic acid complex (2.5 mg/kg) and KMUP-1-γ-polyglutamic acid complex (2.5 mg/kg) are orally administered to the mice for inhibiting the lung fibrosis induced by TGF-β expression in tracheal lavage solution that resulted from 60 mg/kg BM inhalation (Table 6). The state of the TGF-β expression in the lavage is measured by the enzyme immunoassay (EIA).

TABLE 6
The inhibition of the lung fibrosis

| | The changing amount of |
|---|---|
| | TGF-β in the |
| drugs (mg/kg) | lung tissue |
| bleomycin (BM, 60) | |
| BM + Simvastatin (5) | 40 ± 4.3%* |
| BM + KMUP-1 HCl salt (2.5) | 52 ± 5.2%* |
| BM + KMUP-1-Simvastatinic acid (2.5) | 45 ± 4.5%* |
| BM + KMUP-1-Nicotinic acid (2.5) | 42 ± 3.8%* |
| BM + KMUP-1-Glycerrhizic acid | 48 ± 3.8%* |
| BM + KMUP-1-γ-polyglutamic acid (2.5) | 42 ± 1.6%* |
| BM + KMUP-1-GLT Co-polymer acid | 41 ± 1.4%* |

*P < 0.05, which represents the significant difference in comparison of the control group

Anti-inflammatory therapies in the airway are rather ineffective for improving chronic symptoms and reducing inflammation, or reversing the lung function decline and airway remodeling that accompanied a less function in pulmonary vascular system. Specific drug directed against vascular remodeling and chronic inflammation for preventing lung tissue damage and progressive lung function decline is needed. In the present invention, whether the OVA-sensitized and OVA-challenged pulmonary vascular inflammation and remodeling is suppressed by activation of eNOS/cGMP pathway and inhibition of MMP-9 expression remains is investigated. The objective of this invention demonstrates that inhaled KMUP-1 can prevent asthmatic peri-bronchial vascular inflammatory obstruction in lung tissues.

Short Term Nebulization

Short-term KMUP-1 (5 mM, 30 min) nebulization and pretreatment with L-NAME (12 mM, 15 mins) are accomplished using a previously described method. Aerosols were generated by ultrasonic nebulizer from day 21 to 27. Mice were sacrificed on day 28. Three different experiments were performed on cohorts of 7-14 mice per experimental condition.

12. Pulmonary e-NOS Enhancement and MMP-9 Suppression

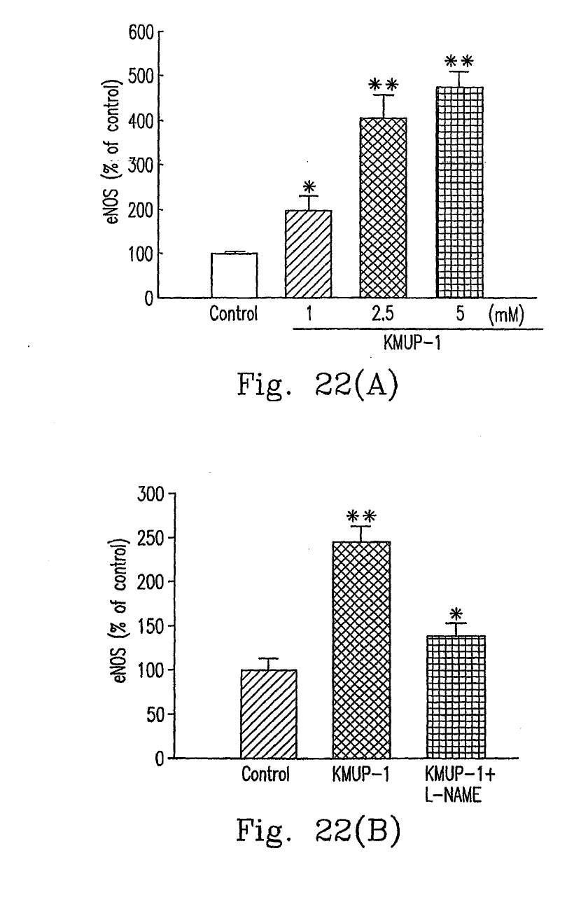

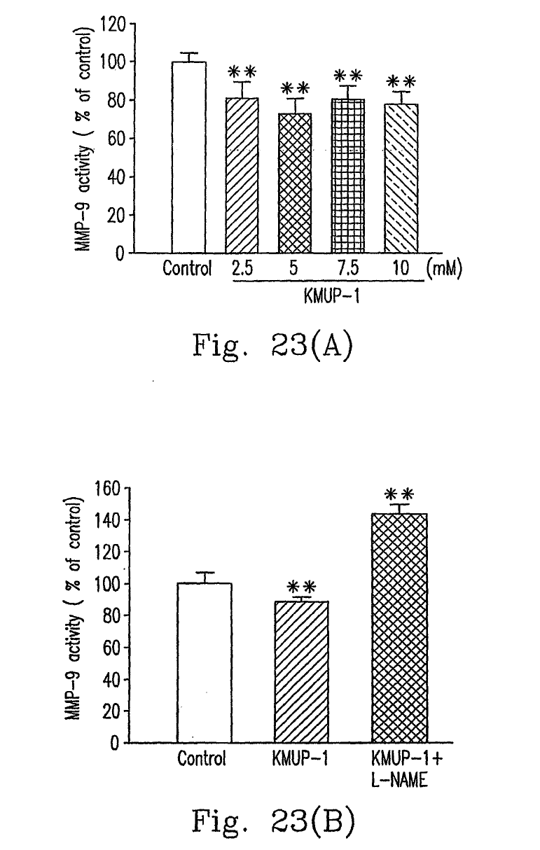

Please refer to FIGS. 22(A) and 23(A), in the short-term experiment without OVA sensitization, KMUP-1 (1.0˜2.5 mM, 30 mins) dose-dependently increased eNOS and decreased MMP-9 expression in mice lung tissues. Please refer to FIGS. 22(B) and 23(B), the changes of eNOS and MMP-9 expression by KMUP-1 (2.5 mM, 30 mins) are reversed by L-NAME-pretreatment (12 mM, 30 mins).

Inhaled KMUP-1 rapidly enters at the airway epithelium and vascular endothelium and thus increases the expression of eNOS, which releases NO and causes a local accumulation of peroxynitrate, reducing OVA-induced increase of MMP-9. This is a reasonable explanation of why KMUP-1 can protect against OVA-sensitized MMP-9 expression. L-NAME-pretreatment can block KMUP-1-induced eNOS expression and MMP-9 suppression, indicating that eNOS is activated earlier than MMP-9 reduction or inactivation. Thus, increased eNOS and restored sGC/PKG by KMUP-1 is suggested to prevent OVA-sensitized MMP-9 expression-associated infiltration of inflammatory cells and NOx production.

13. Pulmonary eNOS, iNOS, sGCα1, PKG and MMP-9 Expression

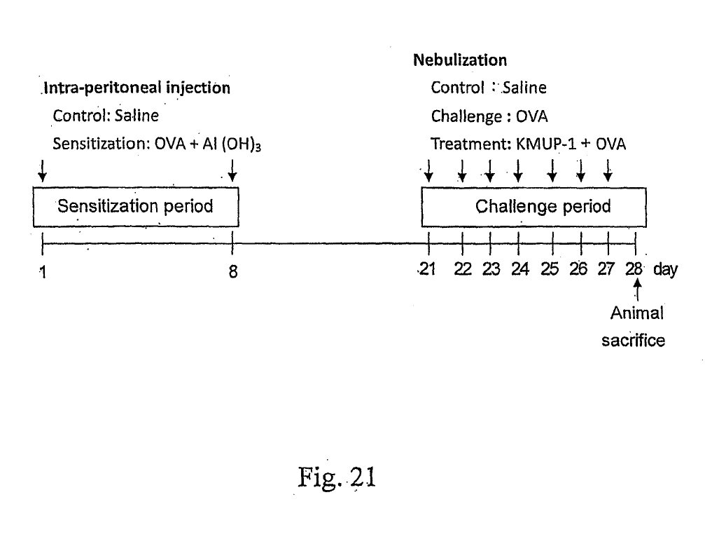

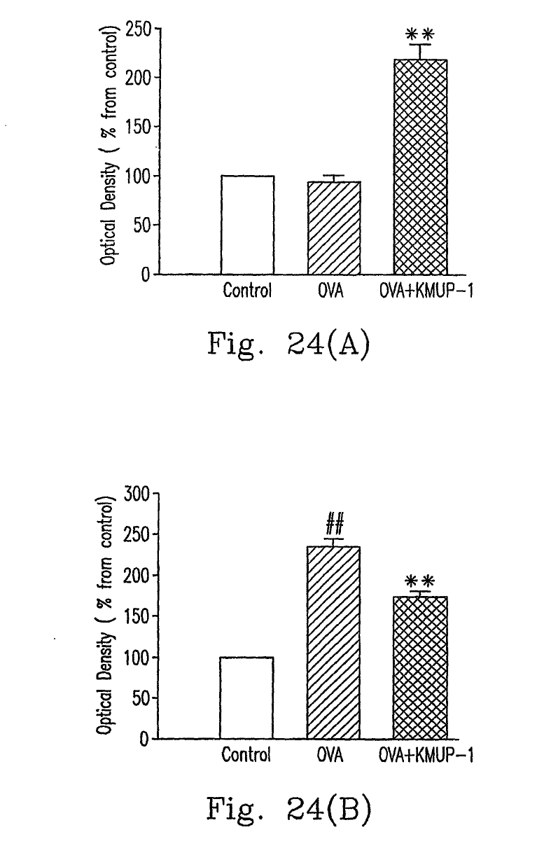

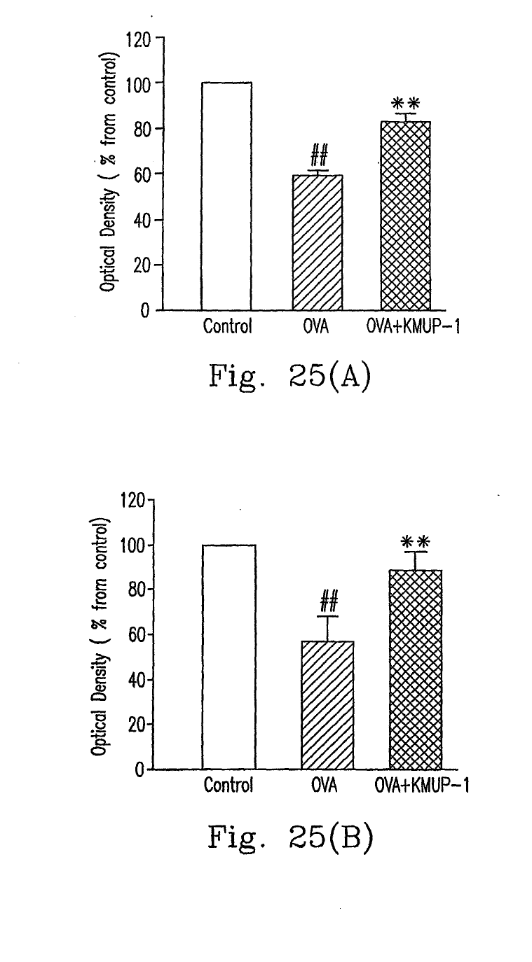

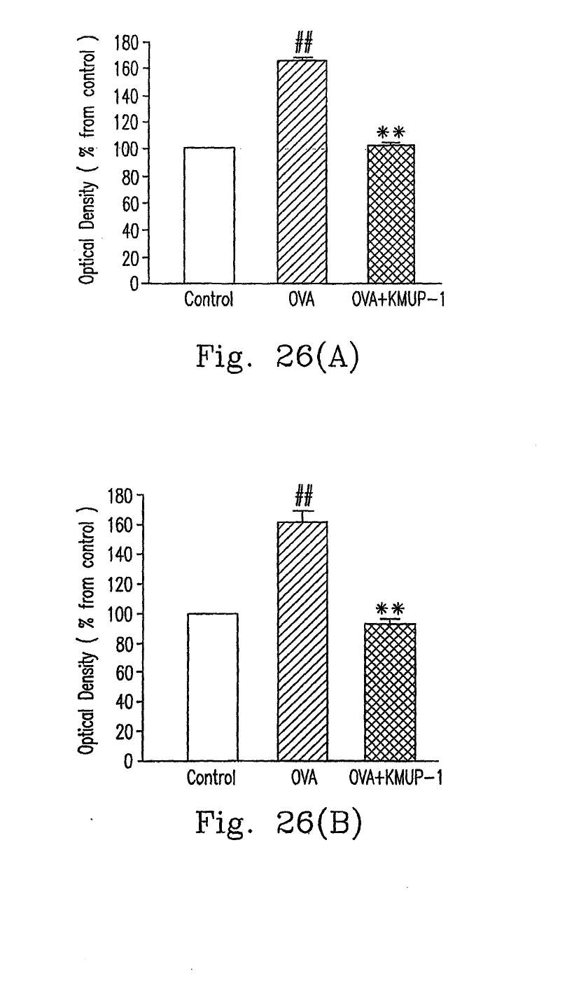

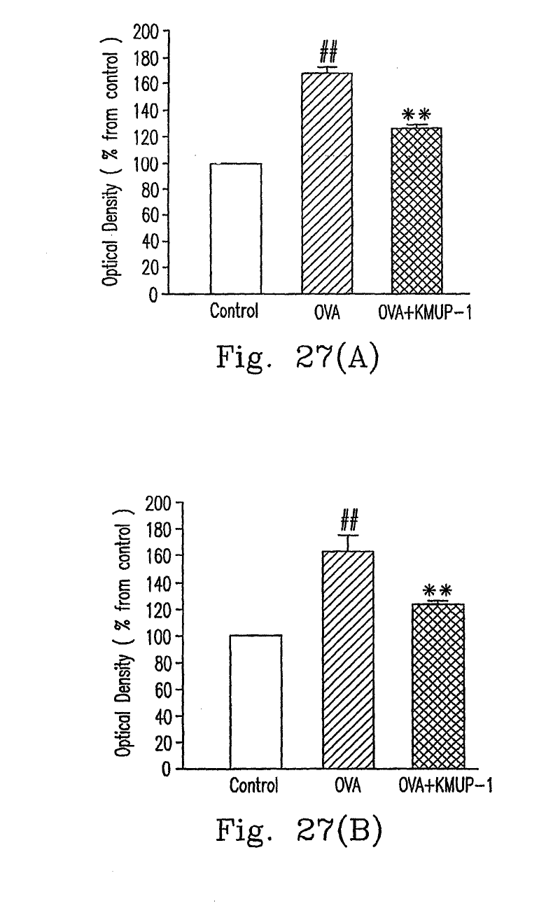

Please refer to FIG. 21, which shows the experimental protocol of the OVA-sensitized and -challenged animal model. In the 28-day experiment, western blotting analysis show that the levels of basal eNOS (FIG. 24A) expression were not affected by OVA, but iNOS (FIG. 24B) is increased and sGCα1 (FIG. 25A), PKG (FIG. 25B), MMP-9 (FIGS. 26A,B), ICAM (FIG. 27A) and VCAM-1 (FIG. 27B) are decreased significantly at 24 hrs after the last OVA-challenge. The increased iNOS and MMP-9 and decreased sGCα1 and PKG expressions by OVA sensitization are significantly reduced by nebulization of KMUP-1. Both expression and its zymography of MMP-9 are decreased. sGCα1 and PKG expressions are significantly reduced by OVA sensitization but prevented by KMUP-1, creating an enhanced NO/sGC/PKG-dependent pathway. Inhaled KMUP-1 can be absorbed into airway and blood flow to increase eNOS and down-regulate Rho-kinase expression by a cGMP-dependent pathway, protecting OVA-sensitized and challenged ICAM-1/VCAM-1 expression-associated cellular adhesion and migration activity of neutrophils.

14. Pulmonary Vascular Wall Thickening Features

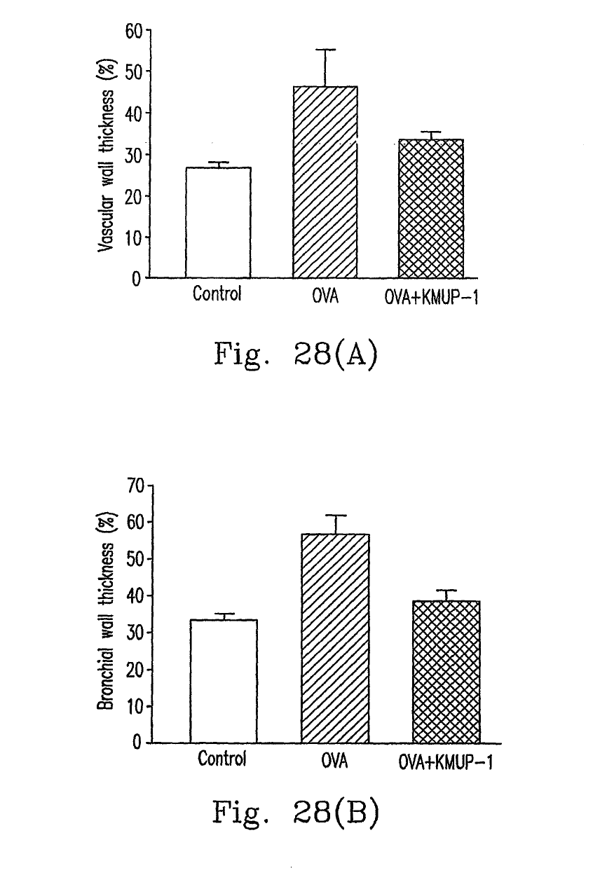

Histological analyses show the typical pathologic features of asthma in the OVA-exposed mice. Numerous inflammatory cells including eosinophils are infiltrated around the peri-vascular and peri-bronchial region, compared to control mice. Mice treated with KMUP-1 show marked reductions of inflammatory cells in the peri-vascular and peri-bronchiolar regions. Please refer to FIG. 28, which shows the effect of KMUP-1 on the vascular (A) and bronchial (B) wall thickness of OVA-sensitized and OVA-challenged mice. After OVA challenge, marked increases in PA wall thickness % are found, compared to controls P<0.01).

OVA-sensitization increases the vascular and bronchial wall thickness, however, treatment with long-term KMUP-1 nebulization prevents these changes. Enhanced eNOS-immunostaining on vascular endothelium indicates that eNOS is the target of KMUP-1 effect. MMP-9-immunostaining in peri-bronchial region and the reduced MMP-9-positive cells in bronchiole co-indicate the reduction of migration of infiltrated inflammatory cells.

15. Immunostaining

OVA decreases vascular eNOS-immunostaining in lung sections, but KMUP-1 prevents this decrease quantitatively. Immunostaining also indicates that MMP-9 is found on inflammatory cells and debris, filling the airway lumen. In control and OVA-sensitized and -challenged mice treated with KMUP-1, MMP-9 positive cells are not found and hardly detected, respectively.

MMP-9 and iNOS expression are induced by OVA sensitization. KMUP-1's anti-inflammatory activity is characterized by their inhibition. Theoretically, OVA sensitization-derived iNOS expression and KMUP-1's eNOS-enhancing activity can co-prevent MMP-9 expression by producing peroxynitrate. However, early eNOS expression by KMUP-1 prevents cytokine-induced iNOS expression. KMUP-1 decreases OVA-induced NOx in BALF, potentially reducing cytokine-induced iNOS expression in infiltrated inflammatory cells, but not in endothelial and epithelial cells. This result is adaptable to the effect of overexpression of eNOS on eosinophilic lung inflammation in eNOS transgenic mice. We presume that KMUP-1 enhances eNOS expression and stabilizes the aberrant sequestration of eNOS in endothelial and epithelial cells. This is why KMUP-1 is favorable to prevent the late worsening of vascular and bronchial inflammation.

16. NOx Levels and Inflammatory Cell Components

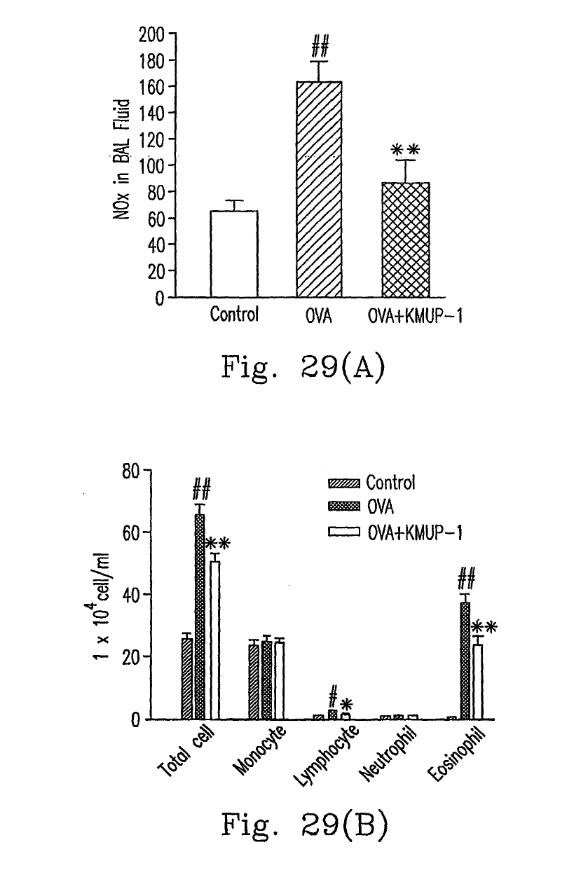

Please refer to FIG. 29, which shows the effects of KMUP-1 on NOx levels (A) and cellular components (B) in BALF. Griess reagent analysis shows that the basal levels of NOx (65.4+8.2 μM) in BALF are significantly increased to 162.8+15.7 μM by OVA at 24 hrs after the last challenge, compared with levels after saline nebulization. The increased levels of NOx (nitrate+nitrite) in BALF are significantly reduced by KMUP-1 to 86.3±17.3 μM.

The numbers of total cells, lymphocytes and eosinophils in BALF are significantly increased at 24 hrs after OVA challenge. KMUP-1 nebulization significantly reduces the increase in total cells, lymphocytes and eosinophils elicited in the airway lumen 24 hrs after the last OVA challenge. Inflammation and remodeling of the bronchioles is associated with infiltration of blood-born lymphocytes, eosinophils and neutrophils, migrating into bronchial lumen. During the course of allergic disease, structural changes occur in the airways, referred to as remodeling. However, treatment with KMUP-1 prevents the migration of blood born cells into bronchiole.

KMUP-1 reduces allergic pulmonary vascular inflammation and remodeling by enhancing eNOS/sGC/PKG and suppressing ICAM-1/VCAM-1 and MMP-9/iNOS expression. KMUP-1 inhalation displays a double anti-inflammation activity on vascular endothelium and bronchial epithelium by enhancing eNOS. KMUP-1 shows considerable promise for treating pulmonary vascular inflammation and remodeling to alleviate asthma, COPD and hypoxic PAH.

### Example 1

### Preparation of KMUP-3HCl Salt (7-[2-[4-(4-nitrobenzene)piperazinyl]ethyl]-1,3-dimethyl xanthine HCl, 1

KMUP-3 (8.3 g) is dissolved in a mixture of ethanol (10 mL) and 1N HCl (60 mL). The solution is reacted at 50° C. for 20 mins, the methanol is added thereinto under room temperature and the solution is incubated over night for crystallization and filtrated to obtain KMUP-3HCl salt (6.4 g).

### Example 2

### Preparation of KMUP-3-Simvastatinic Acid Complex (2)

KMUP-3 (8.3 g) is dissolved in a mixture of ethanol (10 mL) and 1N HCl (60 mL) and reacted at 50° C. for 10 min, the methanol is added thereinto under room temperature and the solution is incubated over night for crystallization and filtrated to obtain KMUP-3HCl (7.4 g). Take KMUP-3HCl salt (9.0 g) and redissolve it in ethanol (150 mL) for use.

In a flask equipped with a magnetic stirrer, simvastatin (4.2 g) dissolved in ethanol (50 ml) is poured, an aqueous solution of sodium hydroxide (4 g/60 ml) and the above-mentioned filtrate of KMUP-3HCl salt are then reacted in the ethanol and kept under room temperature. The mixture is warmed at 50° C. for 20 mins, rapidly filtrated for removing the resulted sodium chloride and then incubated one hour for crystallization to give the KMUP-3-Simvastatinic acid complex (11.8 g).

### Example 3

### Preparation of KMUP-3-Nedocromil mono-sodium Complex (3)

KMUP-3 (8.3 g) is dissolved in a mixture of ethanol (10 mL) and 1 N HCl (60 mL) and reacted at 50° C. for 10 min, the methanol is added thereinto under room temperature and the solution is incubated over night for crystallization and filtrated to obtain KMUP-3HCl (7.4 g). Take KMUP-3HCl salt (9.0 g) and redissolve it in ethanol (150 mL) for use.

In a flask equipped with a magnetic stirrer, nedocromil di-sodium (4.2 g) dissolved in ethanol (50 ml) is poured, to which an aqueous solution and the above-mentioned filtrate of KMUP-3HCl salt reacted with the ethanol are added under room temperature. The mixture is reacted at 50° C. for 20 mins, rapidly filtrated for removing sodium chloride and incubated one hour for crystallization to give the KMUP-3-nedocromil mono-sodium complex (12.7 g).

### Example 4

### Preparation of KMUP-1-Glycyrrizic Acid Complex (4)

KMUP-1 (8 g) is dissolved in a mixture of ethanol (100 mL) and glycyrrizic acid (16.5 g) and reacted at 50° C. for 20 min. The precipitation is filtered under room temperature, the methanol is added thereinto and the solution is incubated over night for re-crystallization and filtrated to obtain KMUP-1 glycyrrizic acid complex (23.2 g).

### Example 5

### Preparation of KMUP-3-cromolyn mono-sodium complex (5)

KMUP-3HCl salt (9.0 g) is dissolved in a mixture of ethanol (100 mL) and water (30 mL), to which cromolyn di-sodium (10.2 g) dissolved in ethyl alcohol (300 mL) is added and the mixture is reacted at 50° C. for 20 mins. After cooling to room temperature, the white precipitate is obtained, to which the methanol is added under room temperature and being incubated over night for re-crystallization. The crystal is filtered to obtain KMUP-3-Cromolyn mono-sodium complex (18.2 g).

### Example 6

### Preparation of KMUP-3-montelukast Complex (6)

KMUP-3HCl salt (9.0 g) is dissolved in a mixture of ethanol (100 mL) and water (30 mL), to which montelukast (11.7 g) dissolved in ethyl alcohol (300 mL) is added and the mixture is reacted at 50° C. for 20 mins. After cooling to room temperature, the white precipitate is obtained, to which the methanol is added under room temperature after the sodium chloride is removed and the reaction is incubated over night for re-crystallization. The crystal is filtered to obtain KMUP-3-montelukast complex (19.8 g).

### Example 7

### Preparation of KMUP-2HCl Salt

(7-[2-[4-(2-methoxybenzene)piperazinyl]ethyl]-1,3-dimethyl xanthine HCl, 7) KMUP-2 (8.0 g) is dissolved in a mixture of ethanol (10 mL) and 1N HCl (60 mL) for reacting at 50° C. for 10 min. The methanol is added into the solution under room temperature and the solution is incubated over night for crystallization. The crystal is filtrated to obtain the precipitate of KMUP-2HCl salt (6.2 g).

### Example 8

### Preparation of KMUP-1-γ-polyglutamate Complex (8)

2 g of sodium γ-polyglutamate is dissolved in water to form a 5% viscous aqueous solution (40 ml). 2 g of KMUP-1HCl salt powder is added to the solution and the mixture is stirred at 50° C. for 1 hr to obtain a white precipitate. The solution is poured out and the ethanol (100 ml) is added for removing sodium chloride and dehydration. Ethanol (100 ml) is added additionally to wash out the unreacted KMUP-1, and the precipitate is dry over night (50° C.) to obtain KMUP-1-γ-polyglutamate complex (2.6 g).

### Example 9

### Preparation of KMUP-1-PGI₂ Complex (9)

KMUP-1HCl salt (880 mg) is dissolved in a mixture of ethanol (100 mL) and water (30 mL), to which PGI₂ sodium (1020 mg) dissolved in ethyl alcohol (300 mL) is added and the mixture for reacting at 50° C. for 20 mins. After cooling to the room temperature, a white precipitate is obtained, to which the methanol is added under room temperature and being incubated over night for re-crystallization. The crystal is filtered to obtain KMUP-1-PGI₂ complex (1620 mg).

### Example 10

### Preparation of KMUP-3-polyglutamate-algininate Complex (10)

Calcium polyglutamate-alginate (2 g) is dissolved in water to form a 5% viscous aqueous solution (40 ml). KMUP-3HCl salt powder (2 g) is added to the solution and the mixture is stirred at 50° C. for 1 hr to obtain a white precipitate. The solution is poured out and the ethanol (100 ml) is added for removing calcium chloride and dehydration. Ethanol (100 ml) is added additionally to wash out the unreacted KMUP-1, and the precipitate is dry over night (50° C.) to obtain KMUP-3-polyglutamate-alginate complex (2.8 g).

### Example 11

### Preparation of KMUP-1-montelukast Complex (11)

KMUP-1HCl salt (8.8 g) is dissolved in a mixture of ethanol (100 mL) and water (30 mL), to which montelukast (11.7 g) dissolved in ethyl alcohol (300 mL) is added and the mixture is reacted at 50° C. for 20 mins. After cooling to room temperature, the white precipitate is obtained, to which the methanol is added under room temperature after the sodium chloride is removed and the reaction is incubated over night for re-crystallization. The crystal is filtered to obtain the precipitate of KMUP-1-montelukast complex (16.8 g).

### Example 12

### Preparation of KMUP-1-L-arginine Complex (12)

KMUP-1 (8.0 g) is dissolved in a mixture of ethanol (10 mL) and 1 N HCl (60 mL) and reacted at 50° C. for 10 min, the methanol is added thereinto under room temperature and the solution is incubated over night for crystallization and filtrated to obtain the precipitate of KMUP-1HCl (6.4 g).

The arginine (3.7 g) is dissolved in the ethanol (100 ml), to which an aqueous solution of sodium hydroxide (4 g/60 ml) is added under room temperature to result in an arginine sodium solution in ethanol for use.

KMUP-1HCl salt (8.8 g) is dissolved in a mixture of ethanol (100 mL) and water (30 mL), to which the arginine sodium solution in hydrated ethanol is added and the mixture is reacted at 50° C. for 60 mins. After cooling to room temperature, the white precipitate is obtained, to which the methanol is added under room temperature after the sodium chloride is removed and the reaction is incubated over night for re-crystallization. The crystal is filtered to obtain the precipitate of KMUP-1-arginine complex (11.6 g).

### Example 13

### Preparation of KMUP-1-L-Etodolac Complex (13)

KMUP-1 (8.8 g) is dissolved in a mixture of ethanol (10 mL) and 1 N HCl (60 mL) and reacted at 50° C. for 10 min, the methanol is added thereinto under room temperature and the solution is incubated over night for crystallization and filtrated for removing sodium chloride and to obtain the precipitate of KMUP-1HCl (6.4 g).

The etodolac (4.9 g) is dissolved in the ethanol (100 ml), to which an aqueous solution of sodium hydroxide (4 g/60 ml) is added under room temperature to result in an etodolac sodium solution in ethanol for use.

KMUP-1HCl salt (8.8 g) is dissolved in a mixture of ethanol (100 mL) and water (30 mL), to which the etodolac sodium solution in hydrated ethanol is added and the mixture is reacted at 50° C. for 60 mins. After cooling to room temperature, the white precipitate is obtained, to which the methanol is added under room temperature after the calcium chloride is removed and the reaction is incubated over night for re-crystallization. The crystal is filtered for removing sodium chloride and to obtain the precipitate of KMUP-1-etodolac complex (12.8 g)

### Example 14

### Preparation of KMUP-1-L-Acetylcystein (14)

The Acetylcysteine (4.9 g) is dissolved in the ethanol (100 ml), to which an aqueous solution of sodium bicarbonate (6 g/60 ml; pH 8.3) is added under room temperature to result in an acetylcystein sodium solution in ethanol for use.

KMUP-1HCl salt (8.8 g) is dissolved in a mixture of ethanol (100 mL) and water (30 mL), to which the acetylcystein sodium solution in hydrated ethanol is added and the mixture is reacted at room temperature for 60 mins. After cooling on ice at 4° C., the white precipitate is obtained, to which the ethanol is added under room temperature after the sodium chloride and CO₂ gas are removed and the reaction is incubated over night for re-crystallization. The crystal is filtered for removing sodium chloride and CO₂ gas to obtain the precipitate of KMUP-1-L-Acetylcystein complex (13.2 g)

### Example 15

### Preparation of the Composition in Tablet

The following components are weighted respectively and filled into a tabletting machine after mixing for preparing tablets.

| | |
|---|---|
| KMUP-3 Montelukast | 1.68 g |
| Lactose | qs |
| Corn starch | qs |

What is claimed is:

1. A pharmaceutical composition for a treatment of an interstitial lung disease, comprising:
an effective amount of an active component being one selected from a group consisting of a KMUP compound, a KMUP monoquaternary ammonium salt and a KMUP monoquaternary ammonium complex salt,
wherein the KMUP monoquaternary ammonium complex salt is synthesized by the KMUP compound and a carboxylic acid derivative of one selected from a group consisting of a statin, a non-steroid anti-inflammatory (NSAIDs) and an anti-asthmatic drug.

2. A pharmaceutical composition as claimed in claim 1, further comprising at least one selected from a group consisting of a pharmaceutically acceptable carrier, a diluent and an excipient.

3. A pharmaceutical composition as claimed in claim 1, wherein the KMUP compound is at least one selected from a group consisting of KMUP-1, KMUP-2 and KMUP-3.

4. A pharmaceutical composition as claimed in claim 1, wherein the KMUP monoquaternary ammonium salt is one of a mineral salt and an organic salt.

5. A pharmaceutical composition as claimed in claim 4, wherein the mineral acid is one selected from a group consisting of HCl, HBr, HI, H₂SO₄, HNO₃, H₃PO₄, NaH₂PO₄ and Na₂HPO₄.

6. A pharmaceutical composition as claimed in claim 4, wherein the organic acid being one selected from a group consisting of a citric acid, a fumaric acid, a maleic acid, a nicotinic acid, an isonicotinic acid, a tartaric acid, a succinic acid, an adipic acid, a fatty acid, a methanesulfonic acid and a phenoxylevulinic acid.

7. A pharmaceutical composition as claimed in claim 1, wherein the interstitial lung disease is caused by an disorder being one selected from a group consisting of an asthma, an inflammatory lung fibrosis, an idiopathic pulmonary fibrosis, an acute pulmonary hypertension and a chronic pulmonary hypertension.

8. A pharmaceutical composition as claimed in claim 1, wherein the KMUP monoquaternary ammonium salt is one selected from a group consisting of KMUP-1-HCl salt, KMUP-2-HCl salt, KMUP-3-HCl salt, KMUP-1-nicotinic acid salt, KMUP-2-nicotinic acid salt and KMUP-3-nicotinic acid salt.

9. A pharmaceutical composition as claimed in claim 1, wherein the KMUP monoquaternary ammonium complex salt is one selected from a group consisting of KMUP-1-glycerrhizic acid salt, KMUP-1-nicotinic acid salt, KMUP-1-folic acid salt, KMUP-1-folinic acid salt, KMUP-1-γ-polyglutamic acid salt, KMUP-1-PGI₂ salt, KMUP-1-GLT co-polymer acid salt, KMUP-1-simvastatinic acid salt, KMUP-1-nedocromil salt, KMUP-1-montelukast salt, KMUP-1-methotrexate salt, KMUP-2-glycerrhizic acid salt, KMUP-2-nicotinic acid salt, KMUP-2-folic acid salt, KMUP-2-folinic acid salt, KMUP-2-γ-polyglutamic acid salt, KMUP-2-PGI₂ salt, KMUP-2-GLT co-polymer acid salt, KMUP-2-simvastatinic acid salt, KMUP-2-nedocromil salt, KMUP-2-montelukast salt, KMUP-2-methotrexate salt, KMUP-3-glycerrhizic acid salt, KMUP-3-nicotinic acid salt, KMUP-3-folic acid salt, KMUP-3-folinic acid salt, KMUP-3-γ-polyglutamic acid salt, KMUP-3-PGI₂ salt, KMUP-3-GLT co-polymer acid salt, KMUP-3-simvastatinic acid salt, KMUP-3-nedocromil salt, KMUP-3-montelukast salt, and KMUP-3-methotrexate salt.

10. A method for treating an interstitial lung disease, comprising:
providing an effective amount of an active component being one of a KMUP compound and a KMUP monoquaternary ammonium salt; and
administering the active component to a subject in need.

11. A method as claimed in claim 10, wherein the interstitial lung disease is caused by an disorder being one selected from a group consisting of an asthma, an inflammatory lung fibrosis, an idiopathic pulmonary fibrosis, an acute pulmonary hypertension and a chronic pulmonary hypertension.

12. A method for treating an interstitial lung disease, comprising:
providing an effective amount of a KMUP monoquaternary ammonium complex salt synthesized by a KMUP compound and a carboxylic acid derivative of one selected from a group consisting of a statin, a non-steroid anti-inflammatory (NSAIDs), an amino acid, an acetylcysteine and an anti-asthmatic drug; and
administering the KMUP monoquaternary ammonium complex salt to a subject in need.

13. A method as claimed in claim 12, wherein the interstitial lung disease is caused by an disorder being one selected from a group consisting of an asthma, an inflammatory lung fibrosis, an idiopathic pulmonary fibrosis, an acute pulmonary hypertension and a chronic pulmonary hypertension.

14. A method for inhibiting a physiological activity of a lung epithelial cell, comprising a step of:
administrating a pharmaceutically effective amount of a compound of 7-[2-[4-(2-chlorophenyl)piperazinyl]-ethyl]-1,3-dimethylxanthine (KMUP-1) to a mammal in need,
wherein the compound is a Rho-kinase inhibitor and being synthesized from xanthine, and the physiological activity is one selected from a group consisting of a proliferation activity, a migration activity, a pro-inflammatory activity and a combination thereof.

15. A method for preparing a pharmaceutical composition, wherein the pharmaceutical composition has an inhibitory effect on one physiological activity of a lung epithelial cell selected from a group consisting of a proliferation, a migration, a pro-inflammatory and a combination thereof, and the pharmaceutical composition contains a compound of 7-[2-[4-(2-chlorophenyl)piperazinyl]-ethyl]-1,3-dimethylxanthine (KMUP-1).
